SEQUENCE LISTING (24 sequences)

SEQ ID NO: 1 - DNA, 429 bp, Mus musculus
caacctccttaaaatgaatttgcctgttcatctcttggtgcttctgttgttctggattcctgcttccagaggtgatgttgtgatgactcaaactccactctccctgcctgtcagctttggagatcaagtttctatctcttgcaggtctagtcagagtcttgcagacagttatgggaacacctatttgtcttggtacctgcacaagcctggccagtctccacagctcctcatctatgggatttccaacagattttctggggtgccagacaggttcagtggcagtggttcagggacagatttcacactcaagatcagcacaattaagcctgaggacttgggaatgtattactgtttacaaggtacacatcagccgctcacgttcggtgctgggaccaagctggagctgaaacgggctgatgctgcaccaac

SEQ ID NO: 2 - protein, 143 aa, Homo sapiens
NLLKMNLPVHLLVLLLFWIPASRGDVVMTQTPLSLPVSFGDQVSISCRSSQSLADSYGNTYLSWYLHKPGQSPQLLIYGISNRFSGVPDRFSGSGSGTDFTLKISTIKPEDLGMYYCLQGTHQPLTFGAGTKLELKRADAAPT

SEQ ID NO: 3 - protein, 26 aa, Mus musculus
DVVMTQTPLSLPVSFGDQVSISCRSS

SEQ ID NO: 4 - protein, 11 aa, Mus musculus
QSLADSYGNTY

SEQ ID NO: 5 - protein, 17 aa, Mus musculus
LSWYLHKPGQSPQLLIY

SEQ ID NO: 6 - protein, 36 aa, Mus musculus
NRFSGVPDRFSGSGSGTDFTLKISTIKPEDLGMYYC

SEQ ID NO: 7 - protein, 9 aa, Mus musculus
LQGTHQPLT

SEQ ID NO: 8 - protein, 17 aa, Mus musculus
FGAGTKLELKRADAAPT

SEQ ID NO: 9 - DNA, 486 bp, Mus musculus
catgagatcacagttctctctacagttactgagcacacaggacctcaccatgggatggaactatatcatcctctttttggtagcaacagctacagaagtccactcccaggtccgactgcagcagcctggggctgaactggtgaagcctggggcttcagtgaagctgtcctgcaaggcttctggctacaccttcaccagctactggatgcactgggtgaaacagaggcctggacaaggccttgagtggattggagagattaatcctagcaccggtcgtactaattacaatgagaggttcaggagcaaggccacactgactgtagacaaattttccaccacagcctacatgcagttcagcagcctgacatctgaggactctgcggtctattactgtgtaggactacgatttgcttactggggccaagggactctggtctctgtctctgcagccaaaacaacacccccatcagtctatccactggcccc

SEQ ID NO: 10 - protein, 162 aa, Mus musculus
MRSQFSLQLLSTQDLTMGWNYIILFLVATATEVHSQVRLQQPGAELVKPGASVKLSCKASGYTFTSYWMHWVKQRPGQGLEWIGEINPSTGRTNYNERFRSKATLTVDKFSTTAYMQFSSLTSEDSAVYYCVGLRFAYWGQGTLVSVSAAKTTPPSVYPLAP

SEQ ID NO: 11 - DNA, 486 bp, Mus musculus
catgagatcacagttctctctacagttactgagcacacaggacctcaccatgggatggaactatatcatcctctttttggtagcaacagctacagaagtccactcccaggtccgactgcagcagcctggggctgaactggtgaagcctggggcttcagtgaagctgtcctgcaaggcttctggctacaccttcaccagctactggatgcactgggtgaaacagaggcctggacaaggccttgagtggattggagagattaatcctagcaccggtcgtactaattacaatgagaggttcaggagcaaggccacactgactgtagacaaattttccaccacagcctacatgcagttcagcagcctgacatctgaggactctgcggtctattactgtgtaggactacgatttgcttactggggccaagggactctggtctctgtctctgcagccaaaacaacagccccatcggtctatccactggcccc

SEQ ID NO: 12 - protein, 162 aa, Mus musculus
MRSQFSLQLLSTQDLTMGWNYIILFLVATATEVHSQVRLQQPGAELVKPGASVKLSCKASGYTFTSYWMHWVKQRPGQGLEWIGEINPSTGRTNYNERFRSKATLTVDKFSTTAYMQFSSLTSEDSAVYYCVGLRFAYWGQGTLVSVSAAKTTAPSVYPLAP

SEQ ID NO: 13 - DNA, 484 bp, Mus musculus
catggggacagttctctctacagttactgagcacacaggacctcaccatgggatggaactatatcatcctctttttggtagcaacagctacagaagtccactcccaggtccgactgcagcagcctggggctgaactggtgaagcctggggcttcagtgaagctgtcctgcaaggcttctggctacaccttcaccagctactggatgcactgggtgaaacagaggcctggacaaggccttgagtggattggagagattaatcctagcaccggtcgtactaattacaatgagaggttcaggagcaaggccacactgactgtagacaaattttccaccacagcctacatgcagttcagcagcctgacatctgaggactctgcggtctattactgtgtaggactacgatttgcttactggggccaagggactctggtctctgtctctgcagccaaaacaacacccccatcagtctatccactggcccc

SEQ ID NO: 14 - protein, 161 aa, Homo sapiens
WGQFSLQLLSTQDLTMGWNYIILFLVATATEVHSQVRLQQPGAELVKPGASVKLSCKASGYTFTSYWMHWVKQRPGQGLEWIGEINPSTGRTNYNERFRSKATLTVDKFSTTAYMQFSSLTSEDSAVYYCVGLRFAYWGQGTLVSVSAAKTTPPSVYPLAP

SEQ ID NO: 15 - protein, 25 aa, Homo sapiens
QVRLQQPGAELVKPGASVKLSCKAS

SEQ ID NO: 16 - protein, 8 aa, Homo sapiens
GYTFTSYW

SEQ ID NO: 17 - protein, 17 aa, Homo sapiens
MHWVKQRPGQGLEWIGE

SEQ ID NO: 18 - protein, 8 aa, Homo sapiens
INPSTGRT

SEQ ID NO: 19 - protein, 38 aa, Homo sapiens
NYNERFRSKATLTVDKFSTTAYMQFSSLTSEDSAVYYC

SEQ ID NO: 20 - protein, 7 aa, Homo sapiens
VGLRFAY

SEQ ID NO: 21 - protein, 11 aa, Homo sapiens
WGQGTLVSVSA

SEQ ID NO: 22 - protein, 420 aa, Homo sapiens
MLHFHYYDTSNKIMEPHRPNVKTAVPLSLESYHISEEYGFLLPDSLKELPDHYRPWMEIANKLPQLIDAHQLQAHVDKMPLLSCQFLKGHREQRLAHLVLSFLTMGYVWQEGEAQPAEVLPRNLALPFVEVSRNLGLPPILVHSDLVLTNWTKKDPDGFLEIGNLETIISFPGGESLHGFILVTALVEKEAVPGIKALVQATNAILQPNQEALLQALQRLRLSIQDITKTLGQMHDYVDPDIFYAGIRIFLSGWKDNPAMPAGLMYEGVSQEPLKYSGGSAAQSTVLHAFDEFLGIRHSKESGDFLYRMRDYMPPSHKAFIEDIHSAPSLRDYILSSGQDHLLTAYNQCVQALAELRSYHITMVTKYLITAAAKAKHGKPNHLPGPPQALKDRGTGGTAVMSFLKSVRDKTLESILHPRG

SEQ ID NO: 23 - protein, 21 aa, Homo sapiens
RDYILSSGQDHLLTAYNQCVQ

SEQ ID NO: 24 - protein, 21 aa, Mus musculus
RDYILASGPGDCLMAYNQCVE

(19) United States
(12) Patent Application Publication
(10) Pub. No.: US 2023/0242674 A1
(43) Pub. Date: Aug. 3, 2023

(54) COMPOSITIONS COMPRISING ANTIBODIES TO HUMAN IDO-2

(71) Applicant: Lankenau Institute for Medical Research, Wynnewood, PA (US)

(72) Inventors: Lisa Laury-Kleintop, Chesterbrook, PA (US); Summer Sedano, Collegeville, PA (US); Kaylend G. Manley, Philadelphia, PA (US)

(21) Appl. No.: 18/004,618

(22) PCT Filed: Jul. 9, 2021
(86) PCT No.: PCT/US2021/041102

§ 371 Date: Jan. 6, 2023

Related U.S. Application Data
(60) Provisional application No. 63/049,819, filed on Jul. 9, 2020.

(51) Int. Cl. C07K 16/40; A61P 37/02

(52) U.S. Cl. CPC C07K 16/40; A61P 37/02; C07K 2317/565; C07K 2317/567

(57) ABSTRACT

Antibodies that specifically or preferentially bind to human IDO2, epitope-binding fragments, and compositions containing one or more of such antibodies or fragments are disclosed, as are uses of these compositions for treating diseases, such as autoimmune disease.

### CROSS-REFERENCE TO RELATED APPLICATIONS

This application claims priority to U.S. Provisional Application 63/049,819 filed Jul. 9, 2020, the entire contents of which being incorporated herein by reference as though set forth in full.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “MLH125P_ST25.txt”, prepared Jul. 7, 2020 and is 12 kb in size.

### BACKGROUND OF THE INVENTION

Indoleamine 2, 3-dioxygenases (IDO1 and IDO2) are tryptophan catabolic enzymes that catalyze the conversion of tryptophan into kynurenine. IDO1 is an extrahepatic enzyme that catabolizes the essential amino acid tryptophan independent of metabolic processing of tryptophan in the liver. IDO1 monotherapy for treatment of ocular disorders is described in the publication WO/2016/100851; see also, U.S. Pat. No. 10,535,035, incorporated by reference herein.

IDO2 is a closely related protein to IDOL Nucleic acid sequences and amino acid sequences of human and mouse IDO2, as well as various isoforms, splice variants and mutant sequences were identified in U.S. Pat. No. 8,436,151, incorporated by reference herein. IDO2 has approximately 45% sequence identity with human IDO1 and shares the structural features of IDO1 that are known to be essential for heme binding and tryptophan catabolic activity. Indeed, the histidine and aspartic acid residues required for heme binding are conserved between IDO1 and IDO2. IDO2 is a candidate therapeutic target for diseases and disorders associated with immune modulation and/or protein misfolding such as cancer, viral infections, and other pathological conditions. See Metz, R. Cancer Res. 2007 August, 76(15): 7082-7), incorporated by reference herein. The activation of IDO2 has distinct and important properties for the regulation of immune responses. U.S. Pat. No. 8,436,151 claims antibodies that are immunologically specific for IDO2 and do not bind to any significant degree to IDO1.

As discussed in US Patent Publication No. US2019/0062452 (also International patent application publication WO2017/019756), incorporated by reference herein, compositions containing at least one anti-IDO2 antibody or fragment thereof and/or anti-IDO2 aptamer are useful for inhibiting, treating, and/or preventing autoantibody related diseases or disorders (e.g., an autoimmune disease) in a subject.

New compositions for treating such autoimmune diseases in a subject by demonstrating additional target selectivity are needed for therapeutic applications.

### SUMMARY OF THE INVENTION

In one aspect, a recombinant, synthetic or monoclonal antibody that binds to a human IDO2 epitope, or a fragment of said antibody that binds a human IDO2 epitope, is provided. The antibody or fragment comprises in one aspect, a heavy chain variable domain sequence that is an amino acid sequence selected from the group consisting of SEQ ID NO: 10, 12 or 14 or an amino acid sequence at least 80% identical to SEQ ID NO: 10, 12 or 14. In another aspect, the antibody or fragment comprises a light chain variable domain sequence that is an amino acid sequence selected from the group consisting of SEQ ID NO:2 or an amino acid sequence at least 80% identical to this sequence. In still another embodiment, truncations and/or modifications of these sequences SEQ ID NO: 2 or 10, 12 or 14 are described. In another aspect, the same recombinant, synthetic or monoclonal antibody binds to both a human IDO2 epitope and a murine IDO2 epitope.

In another aspect, a recombinant, synthetic or monoclonal antibody or recombinant, synthetic, or recombinant peptide construct that binds to a human IDO2 epitope, or a fragment of said antibody that binds a human IDO2 epitope comprises one or more complementarity determining regions (CDRs). In one embodiment, the CDR is the amino acid sequence of SEQ ID NO: 16. In another embodiment, the CDR is the amino acid sequence of SEQ ID NO: 18. In another embodiment, the CDR is the amino acid sequence of SEQ ID NO: 20. In still another embodiment, two or three of these CDRs are present in the antibody or peptide construct.

In still another aspect, the antibody or fragment or recombinant, synthetic, or recombinant peptide construct that binds to a human IDO2 epitope comprises both heavy chain and light chain variable regions from among those SEQ ID Nos identified herein. In a further embodiment, the antibody comprises a heavy chain and light chain sequence of SEQ ID NOs: 10 and 2, referred to as antibody 13-3. In a further embodiment, the antibody comprises a heavy chain and light chain sequence of SEQ ID NOs: 12 and 2, referred to as antibody 18-1. In a further embodiment, the antibody comprises a heavy chain and light chain sequence of SEQ ID NOs: 14 and 2, referred to as antibody 20-10.

In another aspect, a pharmaceutical composition for inhibiting, treating, and/or preventing autoantibody related diseases or disorders (e.g., an autoimmune disease) in a subject in need thereof is provided that comprises at least one antibody or epitope-binding fragment or modification thereof described herein or at least one recombinant, synthetic, or recombinant peptide construct that binds to a human IDO2 epitope and a pharmaceutically acceptable carrier. In another aspect, a pharmaceutical composition for reducing auto-antibody production in a subject in need thereof is provided that comprises at least one antibody or epitope-binding fragment or modification thereof described herein and a pharmaceutically acceptable carrier.

In another aspect, nucleic acid sequences encoding the antibody fragments, and constructs (e.g., vectors or plasmids) containing the coding sequences, as well as compositions containing the nucleic acid sequences, vectors, or plasmids are also provided.

In another aspect, a method for inhibiting, treating, and/or preventing autoantibody related diseases or disorders (e.g., an autoimmune disease) in a subject in need comprises administering at least one antibody or epitope-binding fragment or modification thereof or at least one recombinant, synthetic, or recombinant peptide construct that binds to a human IDO2 epitope described herein and a pharmaceutically acceptable carrier. In one embodiment, an effective amount of a single antibody or epitope binding fragment or modification thereof as described herein is administered. In another aspect, an effective amount of a mixture of antibodies or fragments or constructs described herein is administered.

Methods of generating the antibodies or the peptide constructs or the nucleic acid sequences encoding them, and formulating the pharmaceutical compositions are also provided.

Other aspects and advantages of these methods and compositions are described further in the following detailed description.

### BRIEF DESCRIPTION OF THE FIGURES

FIG. 1 is a graph showing the binding curves of three anti-human IDO2 monoclonal antibodies 13-3, 18-1 and 20-10 described herein and their binding concentrations vs. human IDO2 recombinant protein. Three cloned hybridomas were generated against the human IDO2 amino acids #331-351 (NP 919270; SEQ ID NO: 23). ELISA analysis compares the binding of the anti-human IDO2 antibodies (mAbs) to recombinant human IDO2 protein (100 ng of recombinant human IDO2 protein was used to coat the ELISA plate). Increasing concentrations of mAb were incubated with the coated protein and anti-IDO2 Ig that bound the fixed protein was detected with an anti-mouse IgG Fc-specific HRP secondary antibody and ABTS substrate. Data is presented as Mean±SD. Data indicates that the antibody recognizes human IDO2 protein.

FIGS. 2A and 2B show results of a competitive ELISA that was developed to test the specificity of the antibodies. 100 ng of the recombinant human IDO2 protein was used to coat the ELISA plate. A constant amount (1 μg/ml) of IDO2 antibody was incubated in solution with specified concentrations of soluble peptide. If the antibody interacted with the soluble peptide, the antibody was then unavailable to bind the coated antigen. Anti-IDO2 Ig that bound the fixed protein was detected with an anti-mouse IgG FC-specific HRP secondary antibody and ABTS substrate. Data is presented as Mean±SD. Data indicates that the antibody recognizes human IDO2 protein, as well as the corresponding murine sequence. FIG. 2A is a graph of a competitive ELISA comparing the recognition of the three anti-human IDO2 mAbs of FIG. 1 to the human epitope, plotting absorbance vs. epitope peptide concentration. FIG. 2B is a graph of a competitive ELISA comparing the recognition of the three anti-human IDO2 mAbs of FIG. 1 to the mouse epitope, plotting absorbance vs. epitope peptide concentration.

FIG. 3 is a Western blot analysis using IDO-expressing Tᵣₑₓ cells. All IDO expressed proteins mouse and human IDO 1 and IDO2 contain a V5 tag. Western blot analysis demonstrates the specificity of the anti-human IDO2 antibody for human and mouse IDO2, not recognizing human or murine IDO1. Cell extracts for IDO expressing cells or the parental T-REX line were prepared and fractionated as previously described (PMID: 17671174 & 26717206). Detection of indicated proteins was done using standard blotting methods, HRP conjugated secondary antibodies and chemiluminescence detection reagents.

FIG. 4A is an ELISA analysis of the binding of an anti-human IDO2 mAb compared to anti-mouse IDO2 mAb 4-3. The coated peptide corresponds to the human or murine IDO2 sequence.

FIG. 4B is a Western blot analysis of IDO-expressing cell lines 20-10 detected with an anti-human IDO2 mAb. All IDO expressed proteins contain a V5 tag.

FIG. 5A-5C show that anti-human IDO2 hybridomas 13-3, 18-1, 20-10 have substantially the same Light Chain Variable Regions (LCV). FIG. 5A shows the nucleic acid alignments of the light chain variable regions of three exemplary anti-IDO2 mAbs, 13-13 LCV, 18-1 and 20-10 SEQ ID NO: 1. FIG. 5B shows the amino acid alignments of three exemplary anti-IDO2 mAbs, 13-13, 18-1 and 20-10, which are identical SEQ ID NO: 2. FIG. 5C shows the identity of the FR and CDR regions of the LCV sequences: FR1 SEQ ID NO: 3, CDR1 SEQ ID NO: 4, FR2 SEQ ID NO: 5, CDR2 (GIS), FR3 SEQ ID NO: 6, CDR3 SEQ ID NO: 7, and FR4 SEQ ID NO: 8. Nucleic acid and amino acid alignments were done using the Multiple Sequence Comparison by Log-Expectation MUSCLE-web-based software (https://www.ebi.ac.uk/Tools/msa/muscle). IgBLAST, an analysis tool for immunoglobulin variable domain sequences was used to delineate the Framework Regions (FR) and the Complementarity Determining Regions (CDR) (https://www.ncbi.nlm.nih.gov/igblast/).

FIGS. 6A-6C show the heavy chain variable region (HCV) nucleic acid and amino acid seqs of mAbs 13-3, 18-1 and 20-10. FIG. 6A shows the nucleic acid alignments of the heavy chain variable regions of three exemplary anti-IDO2 mAbs, 13-13 SEQ ID NO: 9, 18-1 SEQ ID NO: 11 and 20-10 SEQ ID NO: 13. FIG. 6B shows the amino acid alignments of three exemplary anti-IDO2 mAbs, 13-13 SEQ ID NO: 10, 18-1 SEQ ID NO: 12 and 20-10 SEQ ID NO: 14. FIG. 6C shows the identity of the FR and CDR regions of the HCV sequences, which are identical for all three antibodies: FR1 SEQ ID NO: 15, CDR1 SEQ ID NO: 16, FR2 SEQ ID NO: 17, CDR2 SEQ ID NO:18, FR3 SEQ ID NO: 19, CDR3 SEQ ID NO: 20, and FR4 SEQ ID NO: 21. The HCV are almost identical but differ at the N or C terminus. These differences are not reflected in the framework regions (FR) and complementarity determining regions (CDRs). Nucleic acid and amino acid alignments were done using the Multiple Sequence Comparison by Log-Expectation MUSCLE-web-based software (https://www.ebi.ac.uk/Tools/msa/muscle). IgBLAST, an analysis tool for immunoglobulin variable domain sequences was used to delineate the Framework Regions (FR) and the Complementarity Determining Regions (CDR) (www.ncbi.nlm.nih.gov/igblast/).

### DETAILED DESCRIPTION OF THE INVENTION

Antibodies, fragments thereof, antibody constructs and pharmaceutical compositions are described herein that recognize human Indoleamine 2,3-Dioxygenase 2 (IDO2). Antibody sequences that recognize human and murine IDO2 are essential for clinical development of a IDO2 targeted biologic. In one embodiment, as described in detail below, hybridoma cell lines were generated that secrete monoclonal antibodies recognizing human IDO2. In one embodiment, the DNA and amino acid sequences of these antibodies are “humanized” for use as a therapeutic biomolecule. As described below, these antibodies, fragments and constructs are useful in the treatment of subjects having autoimmune disease or cancer, among other conditions subject to treatment with anti-IDO2 therapeutics.

As disclosed herein, a recombinant, synthetic and/or monoclonal antibody that binds to a epitope, or a fragment of the antibody that binds a human IDO2 epitope, is provided. In certain embodiments, the antibody is a murine antibody. In other embodiments, the antibody is a human antibody. In still other embodiments, the antibody or fragment is a synthetic construct employing specific variable region CDR sequences in a suitable framework. In certain embodiments, the antibody is a human antibody of an IgG class. In one embodiment, an anti-human IDO2 antibody comprises a heavy chain variable domain sequence that is an amino acid sequence comprising SEQ ID NO: 10 or an amino acid sequence at least 80% identical thereto. In another embodiment, the anti-human IDO2 antibody comprises a heavy chain variable domain sequence that is at least 81%, 82%, 83%, 84%, 85%, 86%, 87%, 88%, 89%, 90% 91%, 92%, 93%, 94%, 95%, 96%, 97%, 98%, 99%, 99.5%, or 100% identical to SEQ ID NO: 10. In another embodiment an anti-human IDO2 antibody comprises a heavy chain variable domain sequence that is an amino acid sequence comprising SEQ ID NO: 12 or an amino acid sequence at least 80% identical thereto. In another embodiment, the anti-human IDO2 antibody comprises a heavy chain variable domain sequence that is at least 81%, 82%, 83%, 84%, 85%, 86%, 87%, 88%, 89%, 90% 91%, 92%, 93%, 94%, 95%, 96%, 97%, 98%, 99%, 99.5%, or 100% identical to SEQ ID NO: 12. In another embodiment an anti-human IDO2 antibody comprises a heavy chain variable domain sequence that is an amino acid sequence comprising SEQ ID NO: 14 or an amino acid sequence at least 80% identical thereto. In another embodiment, the anti-human IDO2 antibody comprises a heavy chain variable domain sequence that is at least 81%, 82%, 83%, 84%, 85%, 86%, 87%, 88%, 89%, 90% 91%, 92%, 93%, 94%, 95%, 96%, 97%, 98%, 99%, 99.5%, or 100% identical to SEQ ID NO: 14. Such antibody or antigen-binding fragment(s) can also include a light chain variable domain sequence that is an amino acid sequence comprising SEQ ID NO: 2 or an amino acid sequence at least 80% identical thereto. In another embodiment, the anti-human IDO2 antibody comprises a light chain variable domain sequence that is at least 81%, 82%, 83%, 84%, 85%, 86%, 87%, 88%, 89%, 90% 91%, 92%, 93%, 94%, 95%, 96%, 97%, 98%, 99%, 99.5%, or 100% identical to SEQ ID NO: 2.

### Components Utilized in the Compositions and Methods

Human IDO2, its amino acid and nucleotide sequences and its isoforms/variants are provided in GenBank Gene ID: 169355 and GenBank Accession Nos. NM 194294.2 and NP 919270.2. An exemplary amino acid sequence of human IDO2 (e.g., 420 amino acids)

MLHFHYYDTS NKIMEPHRPN VKTAVPLSLE SYHISEEYGF LLPDSLKELP DHYRPWMEIA NKLPQLIDAH QLQAHVDKMP LLSCQFLKGH REQRLAHLVL SFLTMGYVWQ EGEAQPAEVL PRNLALPFVE VSRNLGLPPI LVHSDLVLTN WTKKDPDGFL EIGNLETIIS FPGGESLHGF ILVTALVEKE AVPGIKALVQ ATNAILQPNQ EALLQALQRL RLSIQDITKT LGQMHDYVDP DIFYAGIRIF LSGWKDNPAM PAGLMYEGVS QEPLKYSGGS AAQSTVLHAF DEFLGIRHSK ESGDFLYRMR DYMPPSHKAF IEDIHSAPSL RDYILSSGQD HLLTAYNQCV QALAELRSYH ITMVTKYLIT AAAKAKHGKP NHLPGPPQAL KDRGTGGTAV MSFLKSVRDK TLESILHPRG (SEQ ID NO: 22).

An “antibody” or “antibody molecule” is any immunoglobulin, including antibodies and fragments thereof, that binds to a specific antigen. In one embodiment, antibody or antibody molecule contemplates intact immunoglobulin molecules, immunologically active portions of an immunoglobulin molecule, and fusions of immunologically active portions of an immunoglobulin molecule. The antibodies described herein are capable of specifically complexing with, binding to, identifying or detecting an epitope of an antigen, e.g., a human IDO2 epitope. Unless otherwise indicated, the term “antibody” includes, in addition to antibodies comprising two full-length heavy chains (each chain comprising a variable region and a constant region) and two full-length light chains (each chain comprising a variable region and a constant region), modifications, antigen or epitope binding fragments, as well as “antibody mimics” or “antibody equivalents” or muteins thereof. In one embodiment, an “antibody” refers to an intact immunoglobulin, such as an IgA, IgD, IgE, IgG, and IgM, or to an antigen binding portion thereof that competes with the intact antibody for specific binding, unless otherwise specified. In one embodiment, an intact antibody is an IgG1, IgG2, IgG3 or IgG4. Heavy and light chain variable domain sequences and CDRs may be selected from those described herein, including in the Figures and Sequence Listing, e.g., SEQ ID NOs: 1-21. In certain embodiments, the anti-human IDO2 antibody comprises at least one heavy or light chain sequence that is at least 80%, 81%, 82%, 83%, 84%, 85%, 86%, 87%, 88%, 89%, 90% 91%, 92%, 93%, 94%, 95%, 96%, 97%, 98%, 99%, 99.5%, or 100% identical to SEQ ID NOs: 1-21.

As used herein, an “antibody” or “antigen/epitope binding fragment” herein is used in the broadest sense and encompasses various antibody structures, including but not limited to monoclonal antibodies, polyclonal antibodies, multi specific antibodies (e.g., bispecific antibodies), and antibody fragments so long as they exhibit the desired antigen-binding activity. As used herein, the term refers to a molecule comprising at least complementarity-determining region (CDR) 1, CDR2, and CDR3 of a heavy chain and at least CDR1, CDR2, and CDR3 of a light chain, wherein the molecule is capable of binding to antigen. The term antibody includes, but is not limited to, fragments that are capable of binding antigen, such as Fv, single-chain Fv (scFv), Fab, Fab′, and (Fab′)₂. The term antibody also includes, but is not limited to, chimeric antibodies, humanized antibodies, human antibodies, and antibodies of various species such as mouse, cynomolgus monkey, etc.

More specifically, antibody as described herein refers to an anti-human IDO2 antibody or fragment thereof based upon the sequences defined herein. Such an antibody or fragment includes a monoclonal antibody, a synthetic antibody, a recombinant antibody, a chimeric antibody, a humanized antibody, a human antibody, a CDR-grafted antibody, a multispecific binding construct that can bind two or more epitopes, a dual specific antibody, a bi-specific antibody, a multi-specific antibody, an affinity matured antibody, a single antibody chain or an scFv fragment, a diabody, a single chain comprising complementary scFvs (tandem scFvs) or bispecific tandem scFvs, an Fv construct, a disulfide-linked Fv, a Fab construct, a Fab′ construct, a F(ab′)2 construct, an Fc construct, a monovalent or bivalent construct from which domains non-essential to monoclonal antibody function have been removed, a single-chain molecule containing one VL (variable region of light chain), one VH (variable region of heavy chain) antigen-binding domain, and one or two constant “effector” domains optionally connected by linker domains, a univalent antibody lacking a hinge region, a single domain antibody, a dual variable domain immunoglobulin (DVD-Ig) binding protein or a nanobody, or any recombinant versions thereof. Definitions and examples of these types of structures are found in the art and in, e.g., U.S. Pat. No. 9,902,772, incorporated by reference herein.

The term “heavy chain” refers to a polypeptide comprising at least a heavy chain variable region, with or without a leader sequence. In some embodiments, a heavy chain comprises at least a portion of a heavy chain constant region. The term “full-length heavy chain” refers to a polypeptide comprising a heavy chain variable region and a heavy chain constant region, with or without a leader sequence.

The term “heavy chain variable region” refers to a region comprising a heavy chain complementary determining region (CDR) 1, framework region (FR) 2, CDR2, FR3, and CDR3 of the heavy chain. In some embodiments, a heavy chain variable region also comprises at least a portion of an FR1 and/or at least a portion of an FR4. In some embodiments, a heavy chain CDR1 corresponds to Kabat residues 31 to 35; a heavy chain CDR2 corresponds to Kabat residues 50 to 65; and a heavy chain CDR3 corresponds to Kabat residues 95 to 102. See, e.g., Kabat Sequences of Proteins of Immunological Interest (1987 and 1991, NIH, Bethesda, Md.).

The term “light chain” refers to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence. The term “light chain variable region” refers to a region comprising a light chain CDR1, FR2, HVR2, FR3, and HVR3. In some embodiments, a light chain variable region also comprises an FR1 and/or an FR4. In some embodiments, a light chain CDR1 corresponds to Kabat residues 24 to 34; a light chain CDR2 corresponds to Kabat residues 50 to 56; and a light chain CDR3 corresponds to Kabat residues 89 to 97. See, e.g., Kabat Sequences of Proteins of Immunological Interest (1987 and 1991, NIH, Bethesda, Md.).

A “human antibody” as used herein refers to antibodies produced in humans, antibodies produced in non-human animals that comprise human immunoglobulin genes, such as XenoMouse®, and antibodies selected using in vitro methods, such as phage display, wherein the antibody repertoire is based on a human immunoglobulin sequences.

A “chimeric antibody” refers to an antibody in which a portion of the heavy and/or light chain is derived from a particular source or species, while the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species.

A “humanized antibody” refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an Fab, an scFv, a (Fab′)₂, etc.

“Recombinant antibody” refers to an antibody that is expressed from a cell or cell line transfected with one or more expression vectors comprising a coding sequence of the antibody, where said coding sequence is not naturally associated with the cell or naturally occurred in the cell. Said cell may be termed as a host cell. In certain embodiments, the host cell may be a non-human cell or non-human cell line. In certain embodiments, the host cell may be a non-mammalian cell or cell line, for example, an insect cell or cell line, a yeast cell or cell line, or an E coli cell or cell line. In certain embodiments, the host cell may be a mammalian cell or cell line. In certain embodiments, the host cell may be a non-human mammalian cell or cell line. In certain embodiments, the host cell may be a human cell or cell line, for example a human embryonic kidney 293 cells or a hybridoma cell or cell line. In one embodiment, a recombinant antibody has a glycosylation pattern that is different than the glycosylation pattern of an antibody having the same sequence if it were to exist in nature. In one embodiment, a recombinant antibody is expressed in a mammalian host cell which is not a human host cell. Notably, individual mammalian host cells have unique glycosylation patterns.

Methods for producing such antibodies are well-known in the art. Indeed, commercial vectors for certain antibody and antibody fragment constructs are available. The antibody may also be a protein (e.g., a fusion protein) comprising at least one antibody or antibody fragment. In an embodiment, the antibody comprises an Fc region. In certain embodiments, the Fc region of the antibody binds the FyRIIb receptor. In particular embodiments, these anti-antibodies and fragments thereof have a binding affinity (Kₐ) for an epitope of at least 10³ M. In other embodiments, the antigen binding proteins exhibit a Kₐ of at least 10³ M, at least 10⁴ M, at least 10⁵, or at least 10⁶.

As used herein, an “antibody mimic” or an “antibody equivalent” refers to a molecule that specifically bind antigens but that are not structurally related to antibodies (for example, an amino acid sequence, a protein, or a modified or conjugated version thereof). For example, affibodies, i.e., a class of engineered affinity proteins, generally small (˜6.5 kDa) single domain proteins that can be isolated for high affinity and specificity to any given target, aptamers, polypeptide molecules that bind to a specific target, an affilin, an affitin, an affimer, an alphabody, an anticalin, an avimer, a DARPin (designed ankyrin repeat proteins), a Fynomer, a Kunitz domain peptide, a monobody, a peptabody and others known in the art.

As used herein, a “modification” of an amino acid sequence (e.g., antibody or a fragment thereof) comprises an amino acid sequence wherein one or more amino acid residues are inserted into, deleted from, or substituted into the reference amino acid sequence, e.g., any of amino acid sequence encoding the variable light or heavy chains, and/or CDRs of antibodies 13-3, 18-1 or 20-10. One such modification is the replacement of one amino acid in such a sequence, e.g., any of SEQ ID NO: 1 to 21, with a conservative amino acid. Other modifications include, for example, fusion proteins formed by fusing the heavy chain of a selected antibody into an Ig backbone. Still another modification includes an anti-antibody that has been modified via conjugation to another chemical moiety (such as, for example, polyethylene glycol or albumin, e.g., human serum albumin), or a post-translational modification, such as phosphorylation, glycosylation, acylation, acetylation, formylation, alkylation, amidation, arginylation, polyglutamylation, polyglycylation, butyrylation, gamma-carboxylation, polysialylation, malonylation, hydroxylation, iodination, nucleotide addition, phosphate ester or phosphoramidate, propionylation, pyroglutamate formation, S-glutathionylation, S-nitrosylation, 5-sulfinylation, S-sulfonylation, succinylation addition of a succinyl group to lysine, and sulfation. In another embodiment, a modification of any of antibodies 13-3, 18-1 or 20-10 is a single chain human antibody, having a variable domain region from a heavy chain and a variable domain region from a light chain and a peptide linker connecting the heavy chain and light chain variable domain regions.

As used herein, an antibody construct (e.g., an antibody, an antibody heavy chain, an antibody light chain, or any fragment or modification thereof) comprises three Complementarity-Determining Regions (CDRs, also known as HV, hypervariable regions, namely CDR1, CDR2, CDR3, from N-terminal to C-terminal, or 5′ to 3′ when corresponding nucleic acid sequence is referred to), and four framework regions (FRs, namely FR1, FR2, FR3 and FR4, from N-terminal to C-terminal, or 5′ to 3′ when corresponding nucleic acid sequence is referred to). See, e.g., Janeway, Charles A Jr; Travers, Paul; Walport, Mark; Shlomchik, Mark J (2001). Immunobiology: The Immune System in Health and Disease (5 ed.). New York: Garland Science. ISBN 0-8153-3642-X, which is incorporated herein by its entirety. It would be understood that in the antibody construct, CDRs are arranged non-consecutively, not immediately adjacent to each other, and may be separated by an FR. As part of the variable chain in an antibody construct and T cell receptors generated by B-cells and T-cells respectively, CDRs are where an antigen specifically binds.

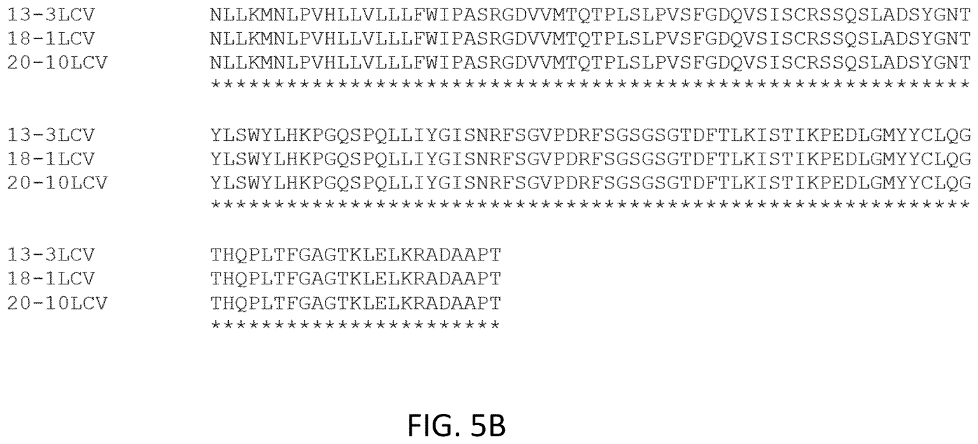

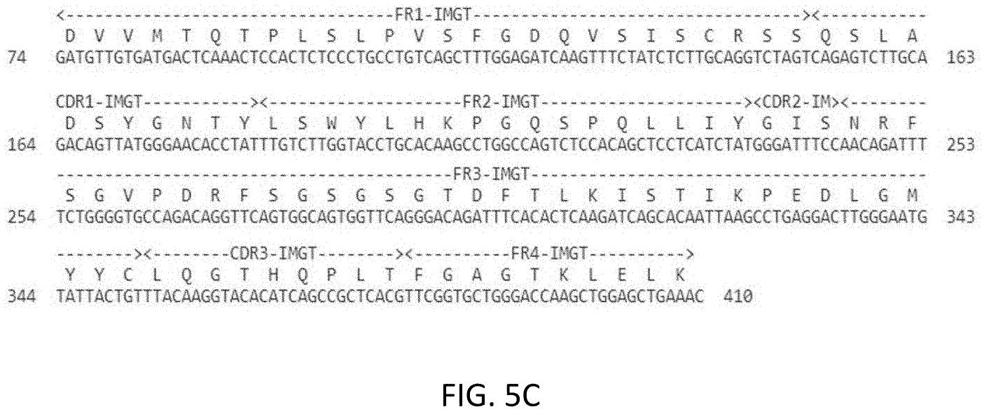

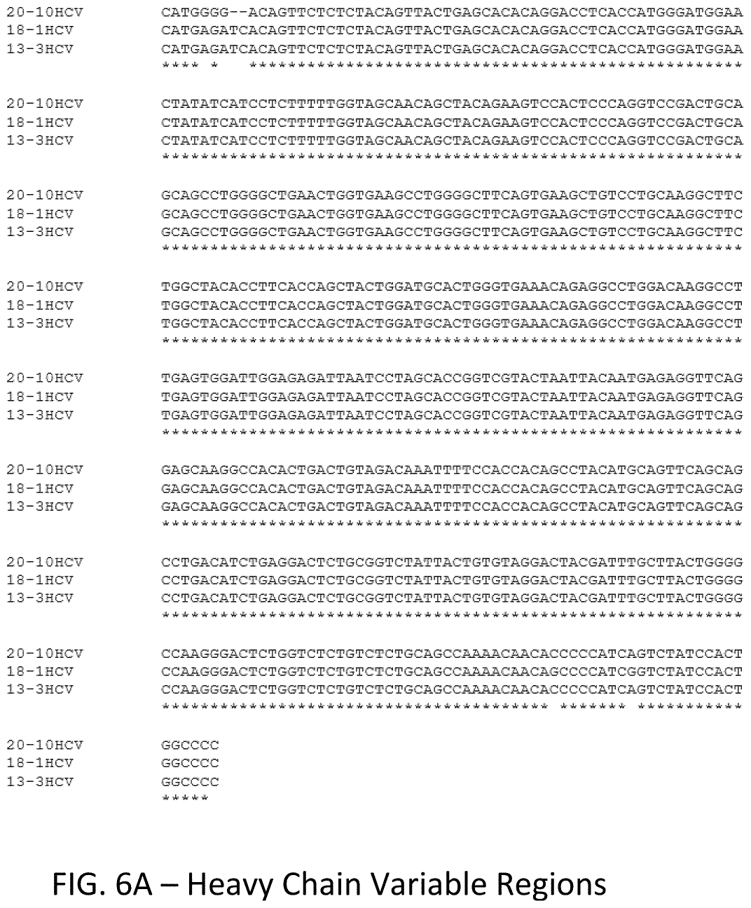

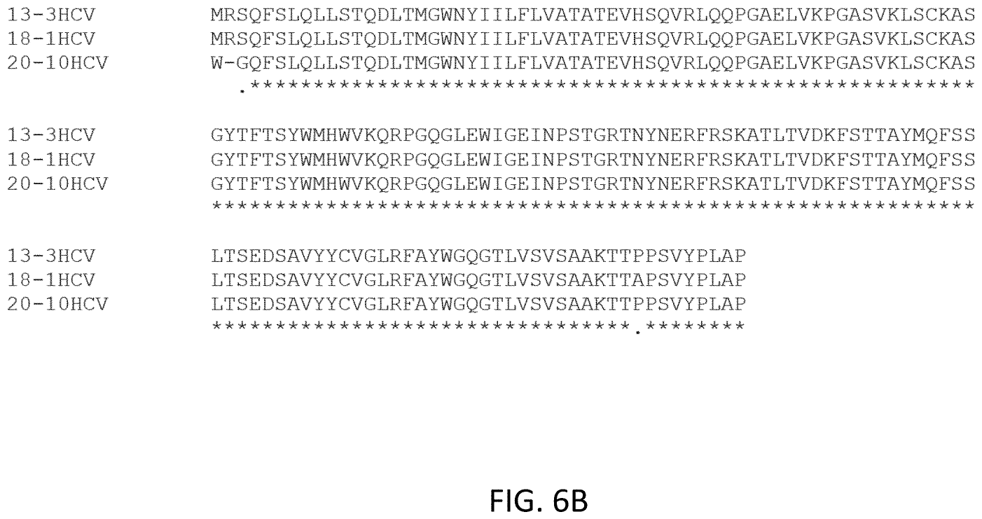

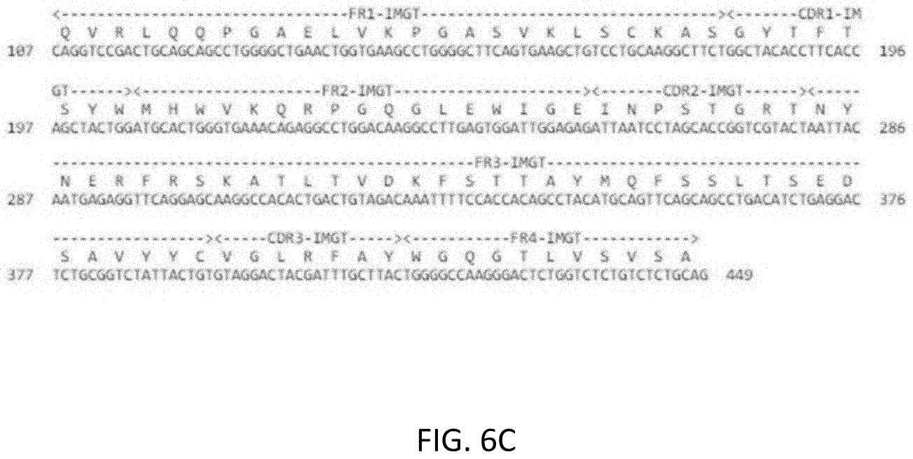

FIGS. 5A to 6C, the incorporated Sequence Listing, and description below provide several examples of antibody constructs via their amino acid sequences, nucleic acid coding sequences, and other regions. Framework regions (FRs) 1 to 4 and CDR 1 to 3 are also identified in the Sequence Listing. Additionally, V-region, D-region and J-region from the perspective of V(D)J recombination can be derived from this information.

V(D)J recombination or rearrangement is a process by which T cells and B cells randomly assemble different gene segments—known as variable (V), diversity (D) and joining (J) genes (or regions, or segments, as used herein)—in order to generate unique receptors (known as antigen receptors) that can collectively recognize many different types of antigens. Briefly, the germ line (unrearranged) genomic DNA configuration of the immunoglobulin heavy chain locus comprises the tandem arrays of V, D, and J gene segments. A germ line kappa or lambda light chain locus comprises unrearranged V and J segments. Stepwise rearrangement of the germ line DNA results in the joining of a heavy chain D and J gene segment, followed by joining of a V segment to the D-J product, to generate the DNA encoding the heavy chain variable region. In the process of rearrangement, the ends of the gene segments are subject to variable amounts of exonuclease digestion and randomized non-templated bases are added at the segment ends, to produce additional sequence diversity at the VDJ junctional region that encodes the complementarity-determining region 3 (CDR3), which is often the region of the antibody heavy chain that has the greatest impact on antigen specificity. A similar process of V and J gene rearrangement with diversification of the VJ junction occurs in the light chain locus, to produce the rearranged light chain gene. See, e.g., Boyd et al, High-Throughput DNA Sequencing Analysis of Antibody Repertoires Microbiology Spectrum. 2. 10.1128/microbiolspec.AID-0017-2014, which is incorporated herein by its entirety.

It would be understood by one of skill in the art that, the antibody constructs, fragments or modifications, and CDR described herein may be used in any embodiment, composition, reagent, or method, including those also described herein. It would also be understood that an antibody, antibody construct, fragment or modification provided herein may comprise an FR or a non-CDR J-region, other than those identified provided in the incorporated Sequence Listing. Such an antibody, antibody construct, fragment or modification may have a binding affinity and/or specificity to its human IDO2 epitope or antigen at about 20%, about 40%, about 50%, about 60%, about 70%, about 80%, about 90%, about 95%, about 97%, about 99%, about 100%, more than about 100%, about 200%, about 300%, or about 500% of that of any antibody constructs described in this specification. Conventional methods, including enzyme-linked immunosorbent assay (ELISA), radioimmunoassay (RIA), MSD assay, and antibody phage display library, may be used to determine such binding affinity and/or specificity.

As used herein, the term “immunologically specific” refers to proteins or polypeptides, particularly antibodies, that bind to one or more epitopes of a protein or compound of interest, but which do not substantially recognize and bind other molecules in a sample containing a mixed population of antigenic biological molecules.

An “epitope” as used herein refers to the portion of a human IDO2 protein or any naturally occurring or synthetic or recombinant amino acid sequence that is capable of specifically complexing with one or more of the antibodies 13-1, 18-1 or 20-10 or fragments or modified antibodies, as described herein. An epitope can comprise non-contiguous portions of the molecule (e.g., in a polypeptide, amino acid residues that are not contiguous in the polypeptide's primary sequence but that, in the context of the polypeptide's tertiary and quaternary structure, are near enough to each other to be bound by an antigen binding protein). Specific embodiments of an anti-human IDO2 antibody are defined in detail below. In one embodiment, the epitope to which an anti-human IDO2 antibody as described herein binds is that of amino acids 331-351 of human IDO2, i.e., the sequence RDYILSSGQDHLLTAYNQCVQ SEQ ID NO: 23. In another embodiment, the epitope to which an anti-human IDO2 antibody also binds is that of amino acids 331-351 of murine IDO2, i.e., the sequence RDYILASGPGDCLMAYNQCVE SEQ ID NO: 24.

The terms “percent (%) identity”, “sequence identity”, “percent sequence identity”, or “percent identical” in the context of amino acid sequences or nucleotide sequences refers to the residues in the two sequences which are the same when aligned for correspondence. Percent identity may be readily determined for amino acid sequences or nucleotide sequences over the full-length of a protein, polypeptide, or encoding region thereof, e.g., about 15 amino acids, about 150 amino acids, or a peptide fragment thereof or the corresponding nucleic acid sequence coding sequences. A suitable amino acid fragment may be at least about 4 amino acids in length and may be up to about 200 or up to about 700 amino acids or nucleotide fragments of from about 12 nucleotides to about 600 to 2100 nucleotides. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Sequence alignment programs are available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., (THOMPSON et al. 1999).

As used herein, the “conservative amino acid replacement” or “conservative amino acid substitutions” refers to a change, replacement or substitution of an amino acid to a different amino acid with similar biochemical properties (e.g. charge, hydrophobicity and size), which is known by practitioners of the art. Also see, e.g. FRENCH et al. 1983, and YAMPOLSKY et al. 2005. In certain embodiments, a CDR of the disclosed antibody or fragment thereof is free of conservative amino acid replacement.

“Patient” or “subject” or “host” as used herein means a male or female mammalian animal, including a human, a veterinary or farm animal, a domestic animal or pet, and animals normally used for clinical research. In one embodiment, the subject of these methods and compositions is a human.

The terms “inhibition” or “inhibit” refer to a decrease or cessation of any event or disease symptom (such as protein ligand binding) or to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. It is not necessary that the inhibition or reduction be complete. For example, in certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

The term “prevent” refers to the prophylactic treatment of a subject who is at risk of developing a condition resulting in a decrease in the probability that the subject will develop the condition.

The term “treat” as used herein refers to any type of treatment that imparts a benefit to a patient afflicted with a disease, including improvement in the condition of the patient (e.g., in one or more symptoms), delay in the progression of the condition, or reduction in severity of the disease and its symptoms.

As used herein, “disease”, “disorder” and “condition” are used interchangeably, to indicate an abnormal state in a subject. As used herein, the term “autoimmune disease” refers to the presence of an autoimmune response (an immune response directed against an auto- or self-antigen) in a subject. Autoimmune diseases include diseases caused by a breakdown of self-tolerance such that the adaptive immune system responds to self-antigens and mediates cell and tissue damage. In a particular embodiment, autoimmune diseases are characterized as being a result of, at least in part, a humoral immune response. In a particular embodiment, the autoimmune disease is T-cell dependent (e.g., T cell help or crosstalk to B cells for autoantibody production). Examples of autoimmune disease include, without limitation: acute disseminated encephalomyelitis (ADEM), acute necrotizing hemorrhagic leukoencephalitis, Addison's disease, agammaglobulinemia, allergic asthma, allergic rhinitis, alopecia areata, amyloidosis, ankylosing spondylitis, antibody-mediated transplantation rejection, anti-GBM/antiTBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenic purpura (ATP), autoimmune thyroid disease, autoimmune urticaria, axonal & neuronal neuropathies, Balo disease, Behcet's disease, bullous pemphigoid, cardiomyopathy, Castleman disease, celiac disease, Chagas disease, chronic fatigue syndrome, chronic inflammatory demyelinating polyradiculoneuropathy (CIDP), chronic recurrent multifocal ostomyelitis (CRMO), Churg-Strauss syndrome, cicatricial pemphigoid/benign mucosal pemphigoid, Crohn's disease, Cogans syndrome, cold agglutinin disease, congenital heart block, coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic fasciitis, erythema nodosum, experimental allergic encephalomyelitis, Evans syndrome, fibromyalgia, fibrosing alveolitis, giant cell arteritis (temporal arteritis), glomerulonephritis, good pasture's syndrome, granulomatosis with polyangiitis (GPA), Graves' disease, Guillain-Barre syndrome, Hashimoto's encephalopathy, Hashimoto's thyroiditis, hemolytic anemia, Henoch Schonlein purpura, herpes gestationis, hypogammaglobulinemia, hypergammaglobulinemia, idiopathic thrombocytopenic purpura (ITP), IgA nephropathy, IgG4-related sclerosing disease, immunoregulatory lipoproteins, inclusion body myositis, inflammatory bowel disease, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosus, ligneous conjunctivitis, linear IgA disease (LAD), lupus (SLE), lyme disease, Meniere's disease, microscopic polyangiitis, mixed connective tissue disease (MCTD), monoclonal gammopathy of undetermined significance (MGUS), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paraneoplastic neurological syndrome, paroxysmal nocturnal hemoglobinuria (PNH), Parry Romberg syndrome, Parsonnage-Turner syndrome, pars planitis (peripheral uveitis), pemphigus (pemphigus vulgaris), peripheral neuropathy, perivenous encephalomyelitis, pernicious anemia, POEMS syndrome, polyarteritis nodosa, type I, II, & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynauds phenomenon, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren syndrome, sperm & testicular autoimmunity, stiff person syndrome, subacute bacterial endocarditis (SBE), Susac's syndrome, sympathetic ophthalmia, Takayasu's arteritis, temporal arteritis/Giant cell arteritis, thrombocytopenic purpura (TTP), Tolosa-Hunt syndrome, transverse myelitis, ulcerative colitis, undifferentiated connective tissue disease (UCTD), uveitis, vasculitis, vesiculobullous dermatosis, vitiligo, Waldenstrom's macroglobulinemia (WM), and Wegener's granulomatosis (Granulomatosis with Polyangiitis (GPA)). In a particular embodiment, the autoimmune disease is selected from the group consisting of rheumatoid arthritis, Type I diabetes, systemic lupus erythematosus, myasthenia gravis, multiple sclerosis, scleroderma, Addison's disease, bullous pemphigoid, pemphigus vulgaris, Guillain-Barre syndrome, Sjogren syndrome, dermatomyositis, thrombotic thrombocytopenic purpura, monoclonal gammopathy of undetermined significance, Waldenstrom's macroglobulinemia, chronic inflammatory demyelinating polyradiculoneuropathy, Hashimoto's encephalapathy, Hashimoto's thyroiditis, Graves' disease, Wegener's granulomatosis, and antibody-mediated transplantation rejection. In a particular embodiment, the autoimmune disease is rheumatoid arthritis. When the autoimmune disease is rheumatoid arthritis, these methods may further comprise the administration of at least one other rheumatoid arthritis therapy (e.g., an anti-inflammatory (e.g., methotrexate), B cell depletion therapy, and/or small molecule inhibitors of the IDO pathway (e.g., 1-methyl-tryptophan).

A “therapeutically effective amount” of a compound or a pharmaceutical composition refers to an amount effective to prevent, inhibit, treat, or lessen the symptoms of a particular disorder or disease. The treatment of an ocular disorder herein may refer to curing, relieving, and/or preventing the ocular disorder, the symptom of it, or the predisposition towards it.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, excipient, auxilliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described, for example, in “Remington's Pharmaceutical Sciences” by E. W. Martin. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described, for example, in “Remington's Pharmaceutical Sciences” by E. W. Martin. In general, the pharmaceutically acceptable carrier of the composition is selected from the group of diluents, preservatives, to solubilizers, emulsifiers, adjuvants and/or carriers. The compositions can include diluents of various buffer content (e.g., Tris-HCl, acetate, phosphate), pH and ionic strength; and additives such as detergents and solubilizing agents (e.g., Tween 80, Polysorbate 80), antioxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polylactic acid, polyglycolic acid, etc., or into liposomes or nanoparticles. Such compositions may influence the physical state, stability, rate of in vivo release, and rate of in vivo clearance of components of the pharmaceutical composition described herein.

The terms “immunosuppressant” and “immunosuppressive agent”, as used herein, include compounds or compositions which suppress immune responses or the symptoms associated therewith. Immunosuppressants include, without limitation, purine analogs (e.g., azathioprine), methotrexate, cyclosporine (e.g., cyclosporin A), cyclophosphamide, leflunomide, mycophenolate (mycophenolate mofetil), steroids (e.g., glucocorticoid, corticosteroid), methylpredni sone, prednisone, non-steroidal anti-inflammatory drug (NSAID), chloroquine, hydroxycloroquine, chlorambucil, CD20 antagonist (e.g., rituximab, ocrelizumab, veltuzumab or ofatumumab), abatacept, a TNF antagonist (e.g., infliximab, adalimumab, etanercept), macrolides (e.g., pimecrolimus, tacrolimus (FK506), and sirolimus), dehydroepiandrosterone, lenalidomide, a CD40 antagonist (e.g., anti-CD40L antibodies), abetimus sodium, BLys antagonists (e.g., anti-BLyS (e.g., belimumab), dactinomycin, bucillamine, penicillamine, leflunomide, mercaptopurine, pyrimidine analogs (e.g., cytosine arabinoside), mizoribine, alkylating agents (e.g., nitrogen mustard, phenylalanine mustard, busulfan, and cyclophosphamide), folic acid antagonsists (e.g., aminopterin and methotrexate), antibiotics (e.g., rapamycin, actinomycin D, mitomycin C, puromycin, and chloramphenicol), human IgG, antilymphocyte globulin (ALG), antibodies (e.g., anti-CD3 (OKT3), anti-CD4 (OKT4), anti-CDS, anti-CD7, anti-IL-2 receptor (e.g., daclizumab and basiliximab), anti-alpha/beta TCR, anti-ICAM-1, muromonab-CD3, anti-IL-12, alemtuzumab and antibodies to immunotoxins), and derivatives and analogs thereof

As used herein, an “anti-inflammatory agent” refers to compounds for the treatment of an inflammatory disease or the symptoms associated therewith. Anti-inflammatory agents include, without limitation, non-steroidal anti-inflammatory drugs (NSAIDs; e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, tramcinolone, and fluticasone), rapamycin, acetaminophen, glucocorticoids, steroids, beta-agonists, anticholinergic agents, methyl xanthines, gold injections (e.g., sodium aurothiomalate), sulphasalazine, and 15 dapsone.

A “cell-penetrating peptide” refers to a peptide which can transduce another peptide, protein, or nucleic acid into a cell in vitro and/or in vivo—i.e., it facilitates the cellular uptake of molecules. Examples of cell penetrating peptides include, without limitation, Tat peptides, penetratin, transportan, and the like.

It should be understood that while various embodiments in the specification are presented using “comprising” language, under various circumstances, a related embodiment is also described using “consisting of” or “consisting essentially of” language. “Comprising” is a term meaning inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention.

With regard to the description of these inventions, it is intended that each of the compositions herein described, is useful, in another embodiment, in the methods of the invention. In addition, it is also intended that each of the compositions herein described as useful in the methods, is, in another embodiment, itself an embodiment of the invention.

It is to be noted that the term “a” or “an”, refers to one or more, for example, “a Target”, is understood to represent one or more Target(s). As such, the terms “a” (or “an”), “one or more,” and “at least one” is used interchangeably herein.

As used herein, the term “about” means a variability of plus or minus 10% from the reference given, unless otherwise specified.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### Antibodies, Fragments and Methods of Producing Antibodies

The production of antibodies or fragments that specifically bind to a human IDO2 epitope, can utilize any of the amino acid sequences of the heavy chain and light chain variable regions, the CDRs, or the nucleotide sequences encoding same, i.e., SEQ ID Nos: 1 to 21, or the other fragments of the antibodies identified herein, including epitope binding fragments, or modifications thereof as described. In one embodiment, polyclonal antibody compositions are typically produced by immunizing a selected mammal, e.g., a primate, rodent, or human, with a peptide/polypeptide composition containing a specific epitope. The selection of high titer, high affinity polyclonal antibodies can be monitored by standard techniques, such as with an enzyme-linked immunosorbent assay and surface plasma resonance. If desired, the polyclonal antibody molecules can be isolated from the mammal, e.g., from the whole blood, plasma or serum, and further purified from the plasma or serum of the immunized mammal by conventional techniques. Conventional harvesting techniques can include plasmapheresis, protein A and G chromatography, among others. Such polyclonal antibody compositions may themselves be employed as pharmaceutical compositions as described herein.

In another embodiment, monoclonal antibodies can be generated to other human IDO2 epitopes by now conventional techniques, using antibody producing cells obtained from the immunized mammals and fused to non-IgG-producing myeloma cells to form hybridomas or from selection from activated immune B cells with extraction by known molecular biological techniques. These monoclonal antibodies can be further used to prepare other forms of antibodies, e.g., chimeric antibodies, humanized antibodies, human antibodies. Other antibody fragments or ligands can be produced by screening phage display libraries, antibody fragments and mixtures thereof. Techniques for generating these types of antibodies and ligands are well-known in the art and the ligands themselves may be generated using the disclosed amino acid sequences of the above-identified epitopes.^{32,35-39}

Chimeric antibodies may similarly be developed using known techniques. Chimeric antibodies are molecules in which different portions are derived from different animal species. Single chain antibodies may also be prepared by conventional methods, such as described in U.S. Pat. Nos. 4,946,778 and 4,704,692 using the variable portions of the polyclonal or monoclonal antibodies produced according to this invention. Antibody fragments, such as the Fab, F(ab)₂ and scFv fragments and libraries thereof may also be employed in generation of the selective anti-TNF monomer-specific antibodies or ligands as described herein.

The production of bi-specific antibodies or ligands that specifically bind to two or more selected epitopes, can employ conventional techniques. See, e.g., Hornig N, Farber-Schwarz A., “Production of bispecific antibodies: diabodies and tandem scFv.”, 2012, Methods Mol Biol., 907:713-27; Speiss, C. et al, “Bispecific antibodies with natural architecture produced by co-culture of bacteria expressing two distinct half-antibodies, Jul. 7, 2013, Nature Biotechnology, 31:753-758; and Jonathan S Martin and Zhenping Zhu, “Recombinant approaches to IgG-like bispecific antibodies”, 2005 Acta Pharmacologica Sinica, 26: 649-658.

The availability of nucleic acid molecules encoding the heavy and light chains of a human IDO2-binding antibody or encoding the CDRs, also enables production of a recombinant antibody, fragment or modification using in vitro expression methods and cell-free expression systems known in the art. In vitro transcription and translation systems are commercially available, e.g., from Promega Biotech (Madison, Wis.) or Gibco-BRL (Gaithersburg, Md.). The antibodies, epitope-binding fragments or modifications thereof may also be produced by expression in a suitable prokaryotic or eukaryotic system. Similarly modifications may be inserted by use of a variety of CRISPR techniques and other related, e.g., zinc finger, methodologies for modifying amino acid and nucleotide sequences. Antibody recombinant engineering techniques are well-taught in the art including in publications, texts and reviews, such as Edwards, W. B., Xu, B., Akers, W., Cheney, P. P., Liang, K., Rogers, B. E., et al. (2008). Agonist-antagonist dilemma in molecular imaging: evaluation of a monomolecular multimodal imaging agent for the somatostatin receptor. Bioconjug. Chem. 19, 192-200; Roque, A. C., Lowe, C. R., and Taipa, M. A. (2004). Antibodies and genetically engineered related molecules: production and purification. Biotechnol. Prog. 20, 639-654; Smith, G. P. (1985). Filamentous fusion phage: novel expression vectors that display cloned antigens on the virion surface. Science 228, 1315-1317; Saeed, AFUH et al, Antibody Engineering for Pursuing a Healthier Future, Front. Microbiol., 28 Mar. 2017; 8:495; Green, M R and Sambrook, J., 2012, Molecular Cloning: A Laboratory Manual, Cold Spring Harbor, N.Y.: Cold Spring Harbor Laboratory Press, all incorporated herein by reference.

### Humanized Antibodies

The term “humanized antibody” generally refers to antibodies which comprise heavy and light chain variable region sequences from a non-human species (e.g., a rabbit, mouse, etc.) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. Another type of humanized antibody is a CDR-grafted antibody, in which at least one non-human CDR is inserted into a human framework. The latter is typically the focus of the present invention.

In particular, the term “humanized antibody” as used herein, is an antibody or a variant, derivative, analog or fragment thereof which immuno-specifically binds to an antigen of interest and which comprises a framework (FR) region having substantially the amino acid sequence of a human antibody and a complementarity determining region (CDR) having substantially the amino acid sequence of a non-human antibody. As used herein, the term “substantially” in the context of a CDR refers to a CDR having an amino acid sequence at least 50, 55, 60, 65, 70, 75 or 80%, preferably at least 85%, at least 90%, at least 95%, at least 98% or at least 99% identical to the amino acid sequence of a non-human antibody CDR. In one embodiment, the humanized antibody has a CDR region having one or more (for example 1, 2, 3 or 4) amino acid substitutions, additions and/or deletions in comparison to the non-human antibody CDR. Further, the non-human CDR can be engineered to be more “human-like” or compatible with the human body, using known techniques. A humanized antibody comprises substantially all of at least one, and typically two, variable domains (Fab, Fab′, F(ab′)₂, F(ab′)c, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, and CH3, or CH1, CH2, CH3, and CH4 of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

The humanized antibody can be selected from any class of immunoglobulins, including IgY, IgM, IgG, IgD, IgA and IgE, and any isotype, including without limitation IgA1, IgA2, IgG1, IgG2, IgG3 and IgG4. The humanized antibody may comprise sequences from more than one class or isotype, and particular constant domains may be selected to optimize desired effector functions using techniques well known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond exactly to either the donor antibody or the consensus framework. In a preferred embodiment, such mutations, however, will not be extensive. Usually, at least 50, 55, 60, 65, 70, 75 or 80%, preferably at least 85%, more preferably at least 90%, and most preferably at least 95%, 98% or 99% of the humanized antibody residues will correspond to those of the parental FR and CDR sequences. In one embodiment, one or more (for example 1, 2, 3 or 4) amino acid substitutions, additions and/or deletions may be present in the humanized antibody compared to the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987). In a family of immunoglobulins, each position in the consensus sequence is occupied by the amino acid occurring most frequently at that position in the family. If two amino acids occur equally frequently, either can be included in the consensus sequence.

Humanized forms of antibodies can be chimeric antibodies that can comprise at least some sequence from a human and at least some sequence from non-human. A humanized antibody can be a monoclonal antibody. It can be constructed with antigen-binding regions (i.e., CDRs) that can be derived, at least in part, from a non-human source. The humanized antibody can contain at least one CDR that can be derived, at least in part, from a non-human source. The CDR can be derived from a light chain of an antibody or a heavy chain of an antibody. The remainder of the variable regions can be derived from a human source. The constant regions can be derived from a human source. A humanized anti-IDO2 antibody can be a human antibody in which a CDR or fragment thereof can be replaced with a CDR or fragment thereof from a non-human anti-IDO2 antibody. Alternatively, a humanized anti-IDO2 antibody can be a non-human anti-IDO2 antibody in which the constant region or fragment thereof can be replaced with a constant region or fragment thereof from a human antibody. For example, a non-human anti-IDO2 CDR or fragment thereof can comprise at least in part a mouse anti-IDO2 CDR or fragment thereof.

The methods for humanizing antibodies can include, for example, humanization uses CDR grafting (Jones et al., Nature 15 321:522 (1986)) and variants thereof, including “reshaping” (Verhoeyen, et al., 1988 Science 239:1534-1536; Riechmann, et al., 1988 Nature 332:323-337; Tempest, et al., Bio/Technol 1991 9:266-271), “hyperchimerization” (Queen, et al., 1989 Proc Natl Acad Sci USA 86:10029-10033; Co, et al., 1991 Proc Natl Acad Sci USA 88:2869-2873; Co, et al., 1992 J Immunol 148:1149-1154), and “veneering” (Mark, et al., B W Metcalf, B J Dalton (Eds.) Cellular adhesion: molecular definition to therapeutic potential. Plenum Press, New York; 1994:291-312). Superhumanization (Tan, et al., 2002 J Immunol 169: 1119-25) is another variant humanization method that can be used to graft non-human CDRs into human germline antibody sequences having similar CDR canonical structures. A humanized antibody can contain one or more amino acid residues introduced into the antibody from a source that is non-human. This non-human amino acid residue can be referred to as an import residue, which can be obtained from a variable domain of a non-human antibody. The import residue can then be used to as a substitute for a hypervariable region amino acid residue of a corresponding residue in a human antibody. Accordingly, such humanized antibodies can be chimeric antibodies. A chimeric antibody can contain substantially less than an intact human variable domain, which can be substituted by the corresponding sequence from a non-human species. A chimeric antibody can also contain an FR residue substituted by a residue from an analogous site in a non-human antibody either to restore the binding of the chimeric antibody to the target, to reduce the chimeric antibody's heterogeneity, or to remove T-cell epitopes.

One method for making humanized antibodies can utilize transgenic non-human animals. The transgenic non-human animals can be genetically engineered to contain one or more humanized immunoglobulin loci that can undergo gene rearrangement and gene conversion in the transgenic non-human animals to produce diversified humanized immunoglobulins.

Another example of a method for making humanized antibodies can utilize a mouse hybridoma cell line. The desired non-human antibody can be expressed by a mouse hybridoma cell line. These cells can be harvested and total RNA can be isolated. Complementary DNA (cDNA) can be generated that codes for the variable domains of the mouse antibody to be humanized. This can be accomplished using polymerase chain reaction (PCR) primers that can hybridize to the 5′ ends of the mouse leader sequences and to the 5′ ends of the mouse constant regions. The light chain and heavy chain variable regions can be cloned. PCR amplification of the cDNA can be accomplished using light and heavy chain specific primers. The PCR product can be cloned directly into a vector. This vector can be transformed into bacteria. The bacteria can be selected for colonies containing the vector with the mouse variable regions. The mouse variable regions can then be modified at the 5′ and 3′ ends using PCR primers to create restriction enzyme sites for convenient insertion into expression vectors, and to incorporate splice-donor sites for RNA splicing of the variable and constant regions. The modified mouse variable regions can be inserted into the FRs of a human antibody. The final vector can encode the CDRs grafted or humanized into the FRs of the human variable region and the human constant region. These vectors can contain human cytomegalovirus enhancer and promoter for transcription, a gene for selection of transformed cells, and the simian virus 40 origin of replication for COS cells. Expression of the humanized antibody can then be accomplished by transfection of mammalian cells.

Alternatively, a fragment for humanizing an antibody can be derived via proteolytic digestion of intact non-human antibodies. A fragment can also be produced by a recombinant host cell by several different methods. For example, an antibody fragment can be isolated from an antibody phage library. A Fab′-SH fragment can be directly recovered from E. coli and then chemically coupled with another Fab′-SH to form a F(ab′)₂ fragments. A F(ab′)₂ fragment can be isolated directly from recombinant host cell culture. Other techniques for the production of antibody fragments will be apparent to the skilled practitioner. An antibody can be a single-chain Fv fragment (scFv). An antibody fragment can also be a linear antibody.

The choice of human domains, both light and heavy, to be used in making the humanized antibodies can be very important to reduce antigenicity. For example, the sequence of the variable domain of a non-human antibody can be screened against an entire library of known human variable-domain sequences. The human sequence that is closest to that of the non-human can then be accepted as the human FR for the humanized antibody. Another method can use a particular FR derived from the consensus sequence of all human antibodies of a particular subgroup of light or heavy chains. The same FR can be used for several different humanized antibodies.

To retain a high affinity for an antigen and other biological properties, a humanized antibody can be prepared by a process of analysis of parental sequences and various conceptual humanized products using three-dimensional models of the parental and humanized sequences. Three-dimensional immunoglobulin models are commonly available and are familiar to those skilled in the art. Computer programs are available that can illustrate and display probable three-dimensional conformational structures of selected candidate immunoglobulin sequences. Inspection of these displays can permit analysis of the likely role of the residues in the functioning of the candidate immunoglobulin sequence. For example, this inspection can permit the analysis of residues that influence the ability of the candidate immunoglobulin to bind its antigen. In this way, FR residues can be selected and combined from the recipient and import sequences so that a desired antibody characteristic, such as increased affinity for a target antigen, can be achieved.

Another exemplary method of producing an anti-IDO2 antibody can comprise selecting a non-human anti-IDO2 antibody having at least one complementarity determining region, selecting a human antibody heavy chain, selecting a human antibody light chain, introducing at least one complementarity determining region or fragment thereof from the non-human anti-IDO2 antibody heavy chain to form a recombinant heavy chain, and introducing at least one complementarity determining region or fragment thereof from the non-human anti-IDO2 antibody into the human antibody light chain to form a recombinant light chain, characterized in that the selection of each of the human antibody heavy and light chains can be determined solely by sequence homology with the non-human anti-IDO2 antibody heavy and light chains, respectively. A humanized antibody can contain an amino acid residue modification that can be different from either the non-human or human amino acid source, where the modification can maintain or improve affinity of the antibody. For example, the antibody variant of interest can have from about one to about five or about seven amino acid substitutions in the recombinant heavy chain sequence. As another example, the antibody variant of interest can have from about one to about five or about seven amino acid substitutions in the recombinant light chain sequence. Such antibody variants can be prepared by affinity maturation.

### Antibody 13-3

Recombinant, synthetic, monoclonal or other antibodies or fragments that bind to one or more human IDO2 epitopes include, in one embodiment a heavy chain variable domain SEQ ID NO: 10 and/or light chain variable domain sequence SEQ ID NO: 2 (antibody 13-3), or sequences at least 80% (for example, about 85%, about 86%, about 87% about 88%, about 89%, about 90%, about 91%, about 92%, about 93%, about 94%, about 95%, about 96%, about 97%, about 98%, about 99%, or 100%) identical thereto. In certain embodiments, antibodies or fragments that bind to one or more human IDO2 epitopes comprise any one, or two, or all three, CDRs of the heavy chain of antibody 13-3. In certain embodiments, antibodies or fragments that bind to one or more epitopes comprise any one, or two, or all three, CDRs of the light chain of antibody 13-3. In certain embodiments, antibodies or fragments that bind to one or more epitopes comprise any one, or two, or three, or four, or five, or all six, CDRs of antibody 13-3. In certain embodiments, provided herein is a nucleic acid sequence encoding the antibodies or fragments thereof as described SEQ ID NO: 9 (HCV) and 1 (LCV). In certain embodiments, the nucleic acid sequence is suitable for expression the antibodies or fragments thereof in a host cell.

Fragments of antibody 13-3 heavy chain nucleotide sequence SEQ ID NO: 9 and amino acid sequence SEQ ID NO: 10 include those sequences identified in the table below:

Heavy Chain Variable Sequence of mAb 13-3

| Feature | Translated AA Sequence | SEQ ID NO: |
|---|---|---|
| FR1 | Gln Val Arg Leu Gln Gln Pro Gly Ala Glu Leu Val Lys | SEQ ID NO: 15 |
| | Pro Gly Ala Ser Val Lys Leu Ser Cys Lys Ala Ser | |
| CDR1 | Gly Tyr Thr Phe Thr Ser Tyr Trp | SEQ ID NO: 16 |
| FR2 | Met His Trp Val Lys Gln Arg Pro Gly Gln Gly Leu Glu | SEQ ID NO: 17 |
| | Trp Ile Gly Glu | |
| CDR2 | Ile Asn Pro Ser Thr Gly Arg Thr | SEQ ID NO: 18 |
| FR3 | Asn Tyr Asn Glu Arg Phe Arg Ser Lys Ala Thr Leu Thr | SEQ ID NO: 19 |
| | Val Asp Lys Phe Ser Thr Thr Ala Tyr Met Gln Phe Ser | |
| | Ser Leu Thr Ser Glu Asp Ser Ala Val Tyr Tyr Cys | |
| CDR3 | Val Gly Leu Arg Phe Ala Tyr | SEQ ID NO: 20 |
| FR4 | Trp Gly Gln Gly Thr Leu Val Ser Val Ser Ala | SEQ ID NO: 21 |

Fragments of antibody 13-3 light chain nucleotide sequence SEQ ID NO: 1 and amino acid sequence SEQ ID NO: 2 include those sequences identified in the table below:

Light Chain Variable Sequence of mAh 13-3

| Feature | Translated AA Sequence | SEQ ID NO: |
|---|---|---|
| FR1 | Asp Val Val Met Thr Gln Thr Pro Leu Ser Leu Pro Val | SEQ ID NO: 3 |
| | Ser Phe Gly Asp Gln Val Ser Ile Ser Cys Arg Ser Ser | |
| CDR1 | Gln Ser Leu Ala Asp Ser Tyr Gly Asn Thr Tyr | SEQ ID NO: 4 |
| FR2 | Leu Ser Trp Tyr Leu His Lys Pro Gly Gln Ser Pro Gln | SEQ ID NO: 5 |
| | Leu Leu Ile Tyr | |
| CDR2 | Gly Ile Ser | — |
| FR3 | Asn Arg Phe Ser Gly Val Pro Asp Arg Phe Ser Gly Ser | SEQ ID NO: 6 |
| | Gly Ser Gly Thr Asp Phe Thr Leu Lys Ile Ser Thr Ile | |
| | Lys Pro Glu Asp Leu Gly Met Tyr Tyr Cys | |
| CDR3 | Leu Gln Gly Thr His Gln Pro Leu Thr | SEQ ID NO: 7 |
| FR4 | Phe Gly Ala Gly Thr Lys Leu Glu Leu Lys Arg Ala Asp | SEQ ID NO: 9 |
| | Ala Ala Pro Thr | |

### Antibody 18-1

Recombinant, synthetic, monoclonal or other antibodies or fragments that bind to one or more human IDO2 epitopes include, in one embodiment a heavy chain variable domain SEQ ID NO: 12 and/or light chain variable domain sequence SEQ ID NO: 12 and 2 (antibody 18-1), or sequences at least 80% (for example, about 85%, about 86%, about 87% about 88%, about 89%, about 90%, about 91%, about 92%, about 93%, about 94%, about 95%, about 96%, about 97%, about 98%, about 99%, or 100%) identical thereto. In certain embodiments, antibodies or fragments that bind to one or more human IDO2 epitopes comprise any one or two or all three CDRs of the heavy chain of antibody 18-1. In certain embodiments, antibodies or fragments that bind to one or more epitopes comprise any one or two or all three CDRs of the light chain of antibody 18-1. In certain embodiments, antibodies or fragments that bind to one or more epitopes comprise any one or two or three or four or five or all six CDRs of antibody 18-1. The light chain variable sequence of 18-1 is the same as the light chain variable sequence of 13-3. Additionally, the heavy chain CDRs and framework regions of 18-1 are the same as those for 13-3 and represented in the table above. In certain embodiments, provided herein is a nucleic acid sequence encoding the antibodies or fragments thereof as described SEQ ID NO: 11 (HCV) and 1 (LCV). In certain embodiments, the nucleic acid sequence is suitable for expression the antibodies or fragments thereof in a host cell.

### Antibody 20-10

Recombinant, synthetic, monoclonal or other antibodies or fragments that bind to one or more human IDO2 epitopes include, in one embodiment a heavy chain variable domain SEQ ID NO: 14 and/or light chain variable domain sequence SEQ ID NO: 2 (antibody 20-10), or sequences at least 80% (for example, about 85%, about 86%, about 87% about 88%, about 89%, about 90%, about 91%, about 92%, about 93%, about 94%, about 95%, about 96%, about 97%, about 98%, about 99%, or 100%) identical thereto. In certain embodiments, antibodies or fragments that bind to one or more human IDO2 epitopes comprise any one or two or all three CDRs of the heavy chain of antibody 20-10. In certain embodiments, antibodies or fragments that bind to one or more epitopes comprise any one or two or all three CDRs of the light chain of antibody 20-10. In certain embodiments, antibodies or fragments that bind to one or more human IDO2 epitopes comprise any one or two or three or four or five or all six CDRs of antibody 20-10. The light chain variable sequence of 20-10 is the same as the light chain variable sequence of 13-3. Additionally, the heavy chain CDRs and framework regions of 20-10 are the same as those for 13-3 and represented in the table above. In certain embodiments, provided herein is a nucleic acid sequence encoding the antibodies or fragments thereof as described SEQ ID NO: 11 (HCV) and 1 (LCV). In certain embodiments, the nucleic acid sequence is suitable for expression the antibodies or fragments thereof in a host cell.

The antibody and antibody fragment thereof that bind to one of more human IDO2 (and possibly murine IDO2) epitopes may comprise at least one domain from the antibodies described above. For example, the antibody or antibody fragment may comprise at least one, two, or three of the heavy chain variable CDR domains of the anti-human IDO2 antibodies 13-3, 18-1 or 20-10. In another embodiment, the antibody or antibody fragment may comprise one, two or three of the light chain variable CDR domains of 13-3, 18-1 or 20-10. In a particular embodiment, the antibody or antibody fragment comprises at least one or both of the heavy and light chain CDR3 domains.

The antibodies and antibody constructs may be further modified from those exemplified. In a particular embodiment, the domains of the antibody or antibody fragment have at least 90%, 95%, 97%, 99%, or 100% homology or identity with the domains present in the anti-monoclonal antibody 13-3, 18-1 and 20-10, and illustrated in the sequences identified in FIGS. 5A-6C, the Sequence Listing, and the tables above. The domains in the tables may be longer or shorter than the domains identified in the tables by about 1, 2, 3, 4, or 5, amino acids, particularly 1 or 2 amino acids, at the N terminus and/or C-terminus of the domain. The domains may be encoded by nucleotide sequences longer or shorter than those in the Sequence listings by 3, 6, 9, 12, or 15 nucleotides, particularly 3 or 6 nucleotides, at the 5′ terminus and/or 3′ terminus of the sequence encoding a domain.

In certain embodiments, the CDR of an antibody can be determined by one of skill in the art, for example, via various databases, software, or algorithms. See, www.imgt.org/. In certain embodiments, the CDRs are illustrated in FIGS. 5C and 6C as well as in the tables herein. In certain embodiments, the CDRs comprises one or two or three or four or five or six more or less amino acids at the N-terminal side and/or C-terminal side of the CDRs as illustrated herein. In certain embodiments, the CDRs are shifted toward the N-terminal side or the C-terminal side by one or two or three or four or five or six or seven or eight or nine or ten amino acid(s) compared to the ones as illustrated in the figures and tables herein.

In one embodiment of a modification, the antibodies may be humanized. In a particular embodiment, the selected sequences of the heavy or light chains of any of the antibodies disclosed herein (or a portion thereof) are inserted into the backbone of an antibody or antibody fragment construct. For example, the variable light domain and/or variable heavy domain of the antibodies described herein may be inserted into another antibody construct. Still another embodiment comprises a fully human Fab antibody fragment having a heavy chain variable domain sequence at least 80, 85, 90, 95 or 99% identity to an amino acid sequence of SEQ ID NO: 10, 12 or 14; or having a light chain variable domain sequence at least 80, 85, 90, 95 or 99% identity to an amino acid sequence of SEQ ID NO: 2 or combinations thereof. Still other modifications of the antibodies are single chain antibodies having a heavy chain variable domain sequence at least 80, 85, 90, 95 or 99% identity to an amino acid sequence of SEQ ID NO: 16, 18 or 20, and a light chain variable domain sequence at least 80, 85, 90, 95 or 99% identity to an amino acid sequence of SEQ ID NO: 2, 4, or 7, and combinations thereof, with a peptide linker connecting the heavy and light chains. These same combinations can be generated by use of the corresponding chain encoding nucleotide sequences or sequences having at least 80, 85, 90, 95 or 99% identity thereto.

In still another embodiment, the antibody comprises a heavy chain CDR sequence and light chain CDR sequence selected from the group consisting of the heavy chain variable domain CDR sequence and the light chain variable domain CDR sequences of the preceding table.

IDO2 is an intracellular protein rather than a cell surface protein, ligand or receptor. Therefore, in one embodiment, the anti-IDO2 antibody may enter cells through the Fc receptor. In certain embodiments, this receptor is the FyRIIb receptor. This mechanism of entry may result in reduced toxicity or side effects if only cells expressing the Fc receptor are susceptible to anti-IDO2 antibodies. Accordingly, in a particular embodiment of the instant invention, the anti-IDO2 antibody fragment comprises an Fc region. In a particular embodiment, the antibody or fragment thereof is conjugated or linked to a cell penetrating peptide, particularly when the antibody fragment does not contain an Fc region. The instant invention also encompasses synthetic proteins which mimic an immunoglobulin. Examples include, without limitation, Affibody® molecules (Affibody, Bromma, Sweden), darpins (designed ankyrin repeat proteins; Kawe et al. (2006) J. Biol. Chem., 281:40252-40263), and peptabodies (Terskikh et al. (1997) PNAS 94:1663-1668). Other mechanisms for internalization of the antibody, including the structural requirements of the antibody for said internalization, are well known to the skilled artisan.

The antibodies of the instant invention may be further modified. For example, the antibodies may be humanized. In a particular embodiment, the hybrid antibodies (or a portion thereof) are inserted into the backbone of an antibody or antibody fragment construct. For example, the variable light domain and/or variable heavy domain of the antibodies of the instant invention may be inserted into another antibody construct. Methods for recombinantly producing antibodies are well-known in the art. Indeed, commercial vectors for certain antibody and antibody fragment constructs are available.

The antibodies of the instant invention may also be conjugated/linked to other components. For example, the antibodies may be operably linked (e.g., covalently linked, optionally, through a linker) to at least one detectable agent, imaging agent, contrast agent, or therapeutic compound (e.g., see above). The antibodies of the instant invention may also comprise at least one purification tag (e.g., a His-tag). The antibody molecules of the invention may be prepared using a variety of methods known in the art. Polyclonal and monoclonal antibodies may be prepared as described in Current Protocols in Molecular Biology, Ausubel et al. eds. Antibodies may be prepared by chemical cross-linking, hybrid hybridoma techniques and by expression of recombinant antibody fragments expressed in host cells, such as bacteria or yeast cells. In one embodiment of the invention, the antibody molecules are produced by expression of recombinant antibody or antibody fragments in host cells. The nucleic acid molecules encoding the antibody may be inserted into expression vectors and introduced into host cells. The resulting antibody molecules are then isolated and purified from the expression system. The antibodies optionally comprise a purification tag by which the antibody can be purified. The purity of the antibody molecules of the invention may be assessed using standard methods known to those of skill in the art, including, but not limited to, ELISA, immunohistochemistry, ion-exchange chromatography, affinity chromatography, immobilized metal affinity chromatography (IMAC), size exclusion chromatography, polyacrylamide gel electrophoresis (PAGE), western blotting, surface plasmon resonance and mass spectroscopy.

Still other antibody modifications employing the SEQ ID Nos disclosed herein, e.g., as taught by the techniques referenced in above-cited US Patent Publication No. US2019/0062452, incorporated by reference herein.

In certain embodiments, the antibodies, and functional fragments, described herein may be constructed by the cell after delivery of nucleic acids encoding said antibodies. In preferred embodiments, the nucleic acids are delivered by a mRNA vaccine as discussed below.

### Pharmaceutical Compositions

Compositions comprising at least one anti-human IDO2 antibody are also described herein. In a particular embodiment, the composition comprises at least one anti-IDO2 antibody or antibody fragment and at least one pharmaceutically acceptable carrier. The composition may further comprise at least one other therapeutic compound for the inhibition, treatment, and/or prevention of the disease or disorder to be treated (see, e.g., hereinabove). Alternatively, at least one other therapeutic compound may be contained within a separate composition(s) with at least one pharmaceutically acceptable carrier. The present invention also encompasses kits comprising a first composition comprising at least one anti-IDO2 antibody or antibody fragment and a second composition comprising at least one other therapeutic compound for the inhibition, treatment, and/or prevention of the disease or disorder to be treated. The first and second compositions may further comprise at least one pharmaceutically acceptable carrier. As explained hereinabove, the compositions of the instant invention are useful for treating autoantibody related diseases or disorders. A therapeutically effective amount of the composition may be administered to the subject. The dosages, methods, and times of administration are readily determinable by persons skilled in the art, given the teachings provided herein.

In yet another aspect, a pharmaceutical composition for the treatment diseases involving IDO2, such as autoimmune diseases referenced herein comprises at least one antibody or epitope-binding fragment as described above and a pharmaceutically acceptable carrier. In one embodiment, the composition contains one or more of antibodies 13-3, 18-1 or 20-10, as described above. In another embodiment, the composition contains a fragment or other of the above-noted modifications, such as humanization, of one or more of these antibodies. In still another embodiment, the pharmaceutical composition comprises one of more of a heavy chain variable domain sequence of SEQ ID Nos: 10, 12 or 14, or a sequence having at least 80%, at least 85%, at least 90%, at least 95% or at least 99% identity to one of the sequences; or having a light chain variable domain sequence at least 80, 85, 90, 95 or 99% identity to an amino acid sequence of SEQ ID NO: 2, or combinations thereof. Other pharmaceutical compositions can be generated by use of the corresponding chain encoding nucleotide sequences SEQ ID NOs: 9, 11, 13 or 1, or sequences having at least 80, 85, 90, 95 or 99% identity thereto, e.g., for delivery in vectors, viruses and the like.

Still another pharmaceutical composition contains a mixture of two or more of the antibodies or epitope binding antibody fragments as described above and a pharmaceutically acceptable carrier. In one embodiment, the composition contains two or more of antibodies 13-3, 18-1 or 20-10 as described above. In one embodiment, the composition contains antibodies 13-3 and 18-1, or modifications or fragments thereof. In one embodiment, the composition contains antibodies 18-1 and 20-10, or modifications or fragments thereof. In one embodiment, the composition contains antibodies 13-3 and 20-10, or modifications or fragments thereof.

In other embodiments the compositions contain additionally epitope binding antibody fragments of 13-3, 18-1 or 20-10.

In one embodiment, the composition contains all three antibodies 13-3, 18-1 and 20-10, as described above. In another embodiment still other anti-human IDO2 antibodies may also be introduced.

In these compositions, the antibodies, fragments or modifications are present in an amount effective to bind a selected human IDO2 epitope and neutralize or inhibit the effect of the IDO2.

In another embodiment, the composition contains a fragment or other of the above-noted modifications of one or more of these antibodies. In still another embodiment, the pharmaceutical composition comprises one of more of a heavy chain variable domain sequence identified herein, one of more of a light chain variable domain sequence identified herein, or a sequence having at least 80%, at least 85%, at least 90% or at least 95% identity to one of these sequences, or a fragment or modification thereof.

In still another embodiment, the pharmaceutical composition comprises an additional anti-human IDO2 antibody other than those described herein, or an antibody fragment or an antibody or antibody fragment that binds another virus. Still other agents which may be included in a pharmaceutical composition as described herein, particularly without limitation, one or more additional immunostimulatory antibodies, such as an anti-PD-1 antibody, an anti-PD-L1 antibody and/or an anti-CTLA-4 antibody. The antibody can be linked to the agent (as an immuno-complex) or can be administered separately from the agent. In the latter case (separate administration), the antibody can be administered before, after or concurrently with the agent or can be co-administered with other known therapies.

Suitable pharmaceutical carriers or excipients for such compositions include, without limitation, the diluent, adjuvant, excipient, auxiliary agent, carrier or vehicle with which an antibody, fragment or modification thereof is administered, as described above.

### Methods of Use

The enzyme indoleamine 2,3-dioxygenase 2 (IDO2) is responsible for driving autoantibody production. In particular, IDO2 drives autoantibody production in a preclinical model of rheumatoid arthritis (RA). As shown in US patent publication No. US2019/0062452, incorporated herein by reference, administration of anti-IDO2 antibodies to the mouse model of RA blocked IDO2 function and alleviated the disease. Anti-IDO2 antibody treatment was effective at reducing autoantibody levels and delaying the onset and attenuating the overall severity of arthritis compared to mice treated with control antibody. The specificity of the antibody was confirmed by the lack of response in mice genetically deficient for IDO2.

The anti-human IDO2 antibodies can be used to reduce autoantibody production and/or treat any disease or disorder that is caused by or exacerbated by the accumulation of an antibody (or antibodies) directed against a patient's own tissues or cells (i.e., autoantibodies)—a common characteristic of autoimmune disease. Such diseases are mediated by the development of autoreactive immune B cells that secrete antibody. While approaches to eradicate B cells or blunt their action have been developed, the use of the anti-IDO2 antibody approach of the instant invention provides a unique manner to limit antibody secretion by the autoreactive B cell. By blunting production of autoimmune antibodies, the methods of the instant invention do not displace disease-specific approaches that may be developed and utilized. For example, therapeutics that alleviate the inflammatory reaction induced by the autoimmune response may be co-administered with the anti-IDO2 antibody therapy to provide a cooperative or even synergistic effect against the disease. Thus, the anti-IDO2 antibody therapeutic methods offer significant advantages in terms of its simplicity and general utility, but also in its unique mechanism and ability to be combined with other therapeutic approaches. Anti-IDO2 antibody technology retards abnormal B cell function in the production of autoimmune antibodies but has not been shown to disrupt normal B cell function after canonical antigenic challenge.

In certain cases, the application of the anti-IDO2 antibody may be similar to other antibody-based therapies which are tolerable despite their non-targeted aspect for disease treatment. Examples include, but are not limited to, the antibody therapies anti-TNFα (infliximab, adalimumab, etanercept), anti-CD20 (rituximab), and anti-BLyS (belimumab), all of which generally blunt inflammation or eradicate B cells or B cell function. For patients that poorly tolerate these therapies, the anti-IDO2 antibody provides another therapeutic option.

The compositions comprising these anti-human IDO2 antibodies may be used in methods for the inhibition, prevention, and/or treatment of an autoimmune disease. The methods comprise administering at least one anti-IDO2 antibody (and/or anti-IDO2 aptamer (see below) to a subject. The methods of the instant invention may further comprise the administration of at least one other therapeutic for the disease being treated. For example, the anti-IDO2 antibodies may be co-administered with an anti-inflammatory agent and/or immunosuppressant. The agents administered to the subject may be contained within a composition comprising at least one pharmaceutically acceptable carrier. When more than one agent is being administered (e.g., anti-IDO2 antibody with an additional therapeutic), the agents may be administered consecutively (before or after) and/or at the same time (concurrently). The agents may be administered in the same composition or in separate compositions.

The compositions described herein are also useful for the inhibition, prevention, and/or treatment of cancers sustained by antibody secretion (e.g., blood tumors (e.g., multiple myeloma) or solid tumors (e.g., wherein antibody secretion contributes to supportive inflammatory processes) (e.g., squamous cell carcinoma 5 (SCC) (Affara et al., Cancer Cell (2014) 25(6):809-821). These methods comprise administering at least one anti-IDO2 antibody to a subject. The methods described herein further comprise the administration of at least one other therapeutic for the cancer being treated. For example, the methods may further comprise the administration of at least one chemotherapeutic agent and/or anti-cancer therapy (e.g., radiation therapy and/or surgery to remove cancerous cells or a tumor (e.g., resection)). The agents administered to the subject may be contained within a composition comprising at least one pharmaceutically acceptable carrier. When more than one agent is being administered (e.g., anti-IDO2 antibody with an additional chemotherapeutic), the agents may be administered consecutively (before or after) and/or at the same time (concurrently). The agents may be administered in the same composition or in separate compositions.

The compositions are also useful for the inhibition, prevention, and/or treatment of antibody-mediated paraneoplastic syndrome. Paraneoplastic syndromes are disorders associated with cancer, not caused by direct invasion, metastasis or consequences of treatment. The methods comprise administering at least one anti-IDO2 antibody to a subject. Examples of antibody mediated paraneoplastic syndrome include, without limitation, stiff person syndrome (e.g., in breast cancer), dermatomyositis (e.g., in breast cancer), opsoclonus myoclonus (e.g., in breast cancer), peripheral encephalomyelitis (e.g., in lung cancer), and retinopathy (e.g., in lung cancer). These methods further comprise the administration of at least one other therapeutic for the paraneoplastic syndrome or cancer being treated. For example, the methods may further comprise the administration of at least one other therapeutic agent. The agents administered to the subject may be contained within a composition comprising at least one pharmaceutically acceptable carrier. When more than one agent is being administered (e.g., anti-IDO2 antibody with an additional therapeutic), the agents may be administered consecutively (before or after) and/or at the same time (concurrently). The agents may be administered in the same composition or in separate compositions.

Compositions and methods for the inhibition, prevention, and/or treatment of antibody-mediated inflammatory diseases also utilize the antibody constructs described herein. The methods comprise administering at least one anti-IDO2 antibody to a subject. Examples of antibody-mediated inflammatory disease include, without limitation, allergic responses, Celiac disease, Crohn's disease, inflammatory bowel disease, rheumatoid arthritis, systemic lupus erythematosus, myasthenia gravis, scleroderma, type I diabetes, monoclonal gammopathy of undetermined significance (MGUS), Sjogren's syndrome, Waldenstrom's macroglobulinemia and Hashimoto's thyroiditis.

The methods further comprise the administration of at least one other therapeutic for the inflammatory disease being treated. For example, the methods may further comprise the administration of at least one other anti-inflammatory agent. The agents administered to the subject may be contained within a composition comprising at least one pharmaceutically acceptable carrier. When more than one agent is being administered (e.g., anti-IDO2 antibody with an additional therapeutic), the agents may be administered consecutively (before or after) and/or at the same time (concurrently). The agents may be administered in the same composition or in separate compositions.

As stated hereinabove, the methods (and compositions) described herein comprise administering at least one antibody or antibody fragment which is immunologically specific for IDO2 (indoleamine 2,3-dioxygenase 2; anti-IDO2 antibody) and/or binds human IDO2 to a subject. In a particular embodiment, the anti-IDO2 antibody is immunologically specific for human IDO2. In a particular embodiment, the anti IDO2 antibody is immunologically specific for human IDO2 to the exclusion of IDO1.

The antibodies, fragments and modifications of the anti-human IDO2 antibodies described herein are useful in methods for treating an IDO2-related disease, such as an autoimmune disease in a subject comprises administering an effective amount of a single antibody or epitope binding fragment or a mixture of antibodies or fragments. In another aspect, a method for treating a subject suspected of having an autoimmune disease or other IDO-2-related disease comprises administering an effective amount of a single antibody or epitope binding fragment or a mixture of antibodies or fragments as described herein.

In another aspect, a method for treating a subject with such a disease comprises administering an effective amount of a single antibody or epitope binding fragment or a mixture of antibodies or fragments as described herein. In another aspect, a method for identifying a subject with such an autoimmune or other IDO2 related disease comprises applying a single antibody or epitope binding fragment or a mixture of antibodies or fragments as described herein to a biological sample of the subject (for example, a blood sample, a serum sample, or a saliva sample).

In one embodiment, the antibody comprises any of antibodies 13-3, 18-1 or 20-10, or fragments or modifications thereof. In another embodiment, the antibody comprises any of the heavy chain sequences of SEQ ID NOs: 10, 12 or 14, an epitope binding fragment or a modification thereof. In another embodiment, the antibody comprises any of the light chain sequences of SEQ ID NOs: 2, an epitope binding fragment or a modification thereof. In yet another embodiment, the antibody is selected from HC/LC pairs of SEQ ID NOs: 10 and 2, 12 and 2, or 14 and 2 or fragments or modifications thereof.

In one embodiment, the antibody and antibody fragment may comprise at least one domain from one or more of the anti-human IDO2 monoclonal antibodies described herein. See FIGS. 5A through 6C for the sequences of the heavy, chain, light chains, CDRs, framework regions, and encoding nucleic acid sequence of these above referenced antibodies. These exemplified sequences can be used to generate the same antibodies or modifications or fragments thereof.

For example, the antibody or antibody fragment may comprise at least one, two, three, four, five, or all six complementarity-determining region (CDR) domains of the anti-human IDO-2 monoclonal antibodies 13-3, 18-1 or 20-10 or humanized or modified versions thereof. In another embodiment, the antibody or antibody fragment may comprise CDRs from these antibodies. In an embodiment, the antibody or antibody fragment comprises at least one or both of the CDR3 domains. In an embodiment, the domains of the antibody or antibody fragment have at least 90%, 95%, 97%, 99%, or 100% homology or identity with the domains present in the anti-human IDO2 antibodies or modifications as discussed above.

Suitable routes of administering the antibody compositions described herein (e.g., human monoclonal antibodies, multi-specific and bispecific molecules and immune-conjugates, fragments or modifications) are in vivo and in vitro are well known in the art and can be selected by those of ordinary skill. In one embodiment, the antibody compositions can be administered by injection (e.g., intravenous or subcutaneous). Suitable dosages of the molecules used will depend on the age and weight of the subject and the concentration and/or formulation of the antibody composition.

In general, regulatory agencies require that a protein reagent to be used as a therapeutic is formulated so as to have acceptably low levels of pyrogens. Accordingly, therapeutic formulations will generally be distinguished from other formulations in that they are substantially pyrogen free, or at least contain no more than acceptable levels of pyrogen as determined by the appropriate regulatory agency (e.g., FDA).

Therapeutic compositions may be administered with a pharmaceutically acceptable diluent, carrier, or excipient, in unit dosage form. Administration may be parenteral (e.g., intravenous, subcutaneous), oral, or topical, or intravenous, or by inhalation, as non-limiting examples. In addition, any gene therapy technique, using nucleic acids encoding the polypeptides of the invention, may be employed, such as naked DNA delivery, recombinant genes and vectors, cell-based delivery, including ex vivo manipulation of patients' cells, and the like. The concentration of the compound in the formulation varies depending upon a number of factors, including the dosage of the drug to be administered, and the route of administration.

The antibodies as described herein will generally be administered to a patient as a pharmaceutical preparation. The term “patient” as used herein refers to human or animal subjects. These antibodies may be employed therapeutically, under the guidance of a physician for the treatment of the indicated disease or disorder. The pharmaceutical preparation comprising the antibody molecules of the invention may be conveniently formulated for administration with an acceptable medium (e.g., pharmaceutically acceptable carrier) such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the agents in the chosen medium may be varied and the medium may be chosen based on the desired route of administration of the pharmaceutical preparation. Except insofar as any conventional media or agent is incompatible with the agents to be administered, its use in the pharmaceutical preparation is contemplated.

The dose and dosage regimen of an antibody according to the invention that is suitable for administration to a particular patient may be determined by a physician considering the patient's age, sex, weight, general medical condition, and the specific condition and severity thereof for which the antibody is being administered. The physician may also consider the route of administration of the antibody, the pharmaceutical carrier with which the antibody may be combined, and the antibody's biological activity.

Selection of a suitable pharmaceutical preparation depends upon the method of administration chosen. For example, the antibodies of the invention may be administered by direct injection into any desired tissue or into the surrounding area. In this instance, a pharmaceutical preparation comprises the antibody molecules dispersed in a medium that is compatible with the target tissue. Antibodies may also be administered parenterally, by intravenous injection into the blood stream, or by subcutaneous, intramuscular or intraperitoneal injection. Pharmaceutical preparations for parenteral injection are known in the art. If parenteral injection is selected as a method for administering the antibodies, steps must be taken to ensure that sufficient amounts of the molecules reach their target cells to exert a biological effect. The lipophilicity of the antibodies, or the pharmaceutical preparation in which they are delivered, may have to be increased so that the molecules can arrive at their target locations. Furthermore, the antibodies may have to be delivered in a cell-targeting carrier so that sufficient numbers of molecules will reach the target cells. Methods for increasing the lipophilicity of a molecule are known in the art. If a small form of the antibody is to be administered, including but not limited to a Fab fragment, a Dab, an scFv or a diabody, it may be conjugated to a second (carrier) molecule such as, but not limited to polyethylene glycol (PEG) or an albumin-binding antibody or peptide to prolong its retention in 0 blood.

Pharmaceutical compositions containing a compound of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous, oral or parenteral. In preparing the antibody in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form in which case solid pharmaceutical carriers are obviously employed. If desired, tablets may be sugar-coated or enteric-coated by standard techniques. For parenterals, the carrier will usually comprise sterile water, though other ingredients, for example, to aid solubility or for preservative purposes, may be included. Injectable suspensions may also be prepared, in which case appropriate liquid carriers, suspending agents and the like may be employed. A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art.

The appropriate dosage unit for the administration of anti-IDO2 antibody molecules may be determined by evaluating the toxicity of the antibody molecules in animal models. Various concentrations of antibody pharmaceutical preparations may be administered to murine models of the disease or disorder and the minimal and maximal dosages may be determined based on the results and side effects as a result of the treatment. Appropriate dosage unit may also be determined by assessing the efficacy of the antibody molecule treatment in combination with other standard drugs. The dosage units of anti-IDO2 antibody molecules may be determined individually or in combination with another treatment.

The pharmaceutical preparation comprising the anti-IDO2 antibody molecules may be administered at appropriate intervals, for example, at least twice a day or more until the pathological symptoms are reduced or alleviated, after which the dosage may be reduced to a maintenance level. The appropriate interval in a particular case would normally depend on the condition of the patient.

The methods of the instant invention may further comprise monitoring the disease or disorder in the subject after administration of the composition(s) described herein to monitor the efficacy of the method.

### Nucleotide Administration

In some aspects, the invention provides methods comprising delivering one or more polynucleotides, such as those encoding one or more of the antibodies described herein, to a host cell. Methods for converting the protein sequences provided herein into nucleotide sequences are well known by those skilled in the art. Conventional viral and non-viral based gene transfer methods can be used to introduce nucleic acids in mammalian cells or target tissues.

Non-viral vector delivery systems include DNA plasmids, RNA, naked nucleic acid, and nucleic acid complexed with a delivery vehicle, such as a liposome. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g., in vitro or ex vivo administration) or target tissues (e.g., in vivo administration).

In certain embodiments, cells producing said antibodies are administered to a patient. In certain preferred embodiments, the nucleic acids encoding said antibodies, or functional fragments thereof, are delivered using mRNA vaccines.

mRNA Vaccines

The information herein above can be applied to an mRNA vaccine that can be administered to a patient using the methods described above. This method comprises producing proteins in vivo via administration of mRNA to live tissues.

The creation and structure of mRNA vaccines are well known by those skilled in the art. See, Rosa, S. S. et al. “mRNA vaccines manufacturing: Challenges and bottlenecks.” Vaccine 39 (2021): 2190-2200. Construction of mRNA vaccines requires the insertion of the encoded antigen in a DNA template from where the mRNA is transcribed in vitro. Unlike DNA, mRNA only needs to reach the cytosol, where it will be transcribed into the antigen in vivo, using the cell machinery. This way, any desired sequence can be designed, produced in vitro, and delivered to any type of cell.

The structure of mRNA vaccines is similar to eukaryotic mRNA—a single-stranded molecule with a cap at the 50 end, a poly(A) tail at the 30 end and an open reading frame (ORF) flanked by untranslated regions (UTR). The 50 cap is an important component as it enables the translation initiation by binding to a eukaryotic translation initiation factor (eIF4E). Different structures are possible for the 50 cap. The Cap 0 structure, which features a methyl-7 guanine nucleotide linked to the 50 position through a 50 triphosphate, is the simplest. The Cap 1 structure is achieved by the methylation of the mRNA first nucleotide at the ribose 20-0 position. Both caps can be added during in vitro mRNA transcription using a synthetic cap analogue or the proprietary Cap dinucleotide CleanCap®. Another capping approach uses a post-transcription enzymatic reaction based on the vaccinia capping system. This modification brings with it a number of advantages as it improves the translation initiation by recruiting translation initiation factors, protects the synthetic mRNA against exonuclease degradation, and avoids an innate immunity overactivation response. The addition of a 30 poly(A) tail also improves mRNA stability and translational activities, as it protects mRNA from nuclease degradation by the poly(A)-binding protein (PABP). This tail can be added to the transcript by inserting a poly(A) sequence in the DNA template or by an enzymatic reaction. Tail size optimization is an important factor for the stabilization and expression of mRNA. Longer poly-A tails can improve mRNA stability and translation. However, this effect is not linear, and the best tail size is dependent on cell type. The untranslated regions (UTRs) are responsible for the transcription regulation and mRNA stability. These regions strongly affect translation efficiency as the sequences used are involved in the translation machinery recognition, recruitment, and mRNA trafficking. Strategies to modulate the innate immune response, such as the introduction of unnatural nucleosides (NTPs), and to improve translation efficiency, by using codon optimization, are also commonly used in mRNA production.

Two forms of mRNA structure are being extensively studied for vaccine applications: conventional or non-replicating mRNA and self-amplifying mRNA. In the conventional mRNA form, the antigen of choice is only flanked by UTR regions, a 30 poly(A) tail and a 50 cap. This form presents several advantages—molecules are simple and small, and the possibility of unwanted immune response is lowered since no other proteins are encoded. However, this mRNA expression is limited to its transient nature, and higher mRNA doses may be necessary to achieve high expression. Efforts have been made to overcome this bottleneck by using sequence optimization and formulation. Self-amplifying mRNA (saRNA) is based on the addition of a viral replicase gene to enable the mRNA to self-replicate. Usually, sequences of single-stranded RNA viruses, such as alphaviruses, flaviviruses, and picornaviruses, are used. Upon cytoplasm delivery, this type of mRNA produces high levels of the antigen of interest. Despite the use of viral genes, no viral infectious particles or virus-like-particles are observed during expression, reducing the safety concerns. Evaluation of an saRNA vaccine for protection of mouse models against H1N1/PR8 infection showed that a 64-fold lower dose was required to induce an immunologic response when compared with the conventional mRNA vaccine counterpart.

Trans-amplifying mRNA (taRNA) is a new structural modality of mRNA vaccines. The taRNA results from the splitting of the self-amplifying mRNA in a system with two templates, one containing the gene of interest and a second containing the replicase system. The amplification is performed in trans by the replicase in the cyto-plasm. This system presents some advantages over saRNA since it is safer, more versatile and cost-effective to manufacture, as the production of shorter RNAs with high yield and high quality is less challenging. taRNA has already been used to protect mice against influenza with results showing induction of antibodies and protection.

### Delivery

mRNA must cross the cell membrane to reach the cytosol. This is challenging due to the negative charge of the molecule, its relatively large size (300-5000 kDa) and degradability, which can hamper its passive pass through the cell membrane. To overcome this, mRNA can be delivered using different strategies including: i) direct injection of naked mRNA; ii) conjugation with lipid-based carriers, polymers, or peptides; iii) via transfection of dendritic cells (DC).

The induction of an immune response by injection of naked mRNA in conventional and self-amplifying forms has been widely reported. However, mRNA delivery can be limited by the presence of extracellular exonucleases in the target tissues, inefficient cell uptake or unsuccessful endosomal release. Liposomes or lipid nanoparticles (LNPs) are one of the most promising mRNA delivery tools. For example, LNP-mediated delivery of mRNA vaccines against Zika and influenza has shown encouraging results. Although less explored, polymer-based delivery systems can also be used. Polyethylenimine (PEI) systems were successfully implemented as a strategy to deliver mRNA to cells, and intranasally. Additionally, PEI-based systems improved the response to sa-mRNA vaccines in skin explants and in mice. Peptide-based delivery is a less explored system, as only protamine has been evaluated in clinical trials. New delivery approaches include the use of cationic cell-penetrating peptides (CPPs) and anionic peptides. CPPs systems have proved to improve T-Cell immunity response in vivo, modulate innate immune response and enhance protein expression in both DC and human cancer cells in vitro. mRNA polyplexes conjugated with an anion peptide, exhibited an increase in cellular uptake without inducing cytotoxicity in DC cells.

### Kits and Articles of Manufacture

Any of the aforementioned products can be incorporated into a kit which may contain, an oligonucleotide, a polypeptide, a peptide, an antibody, a detectable label, marker, reporter, a pharmaceutically acceptable carrier, a physiologically acceptable carrier, instructions for use, a container, a vessel for administration, an assay substrate, or any combination thereof.

### EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein.

### Example 1—Development of Antibodies Recognizing Human IDO2

Three cloned hybridomas were generated against the human IDO2 amino acids #331-351 (NP 919270; SEQ ID NO: 23). ELISA analysis compares the binding of the anti-human IDO2 antibodies (mAbs) to recombinant human IDO2 protein (100 ng of recombinant human IDO2 protein was used to coat the ELISA plate). Increasing concentrations of mAb were incubated with the coated protein and anti-IDO2 Ig that bound the fixed protein was detected with an anti-mouse IgG Fc-specific HRP secondary antibody and ABTS substrate.

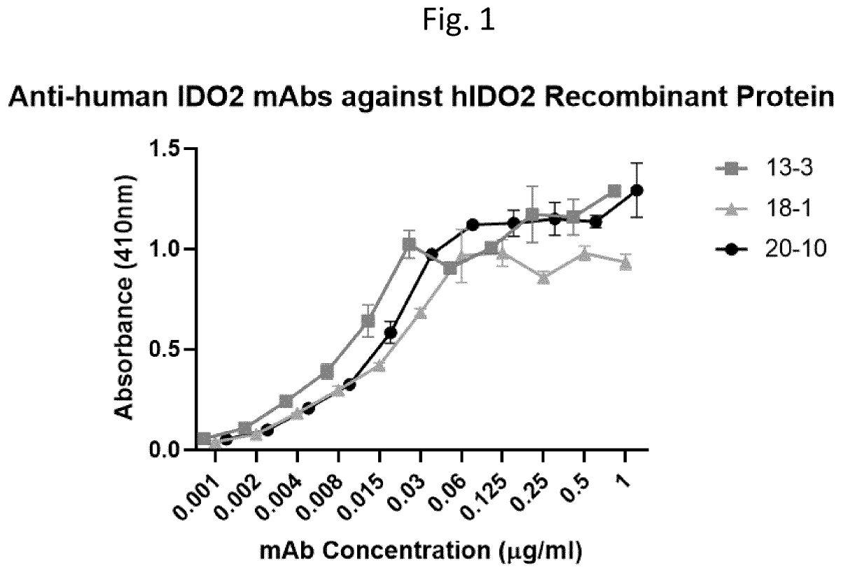

The results are shown in FIG. 1, which is a graph showing the binding curves of three anti-human IDO2 monoclonal antibodies 13-3, 18-1 and 20-10 and their binding concentrations vs. human IDO2 recombinant protein. These data indicate that the antibodies recognize human IDO2 protein.

### Example 2—Specificity of the Antibodies

A competitive ELISA was developed to test the specificity of the antibodies. 100 ng of the recombinant human IDO2 protein was used to coat the ELISA plate. A constant amount (1 μg/ml) of IDO2 antibody was incubated in solution with specified concentrations of soluble peptide. If the antibody interacted with the soluble peptide, the antibody was then unavailable to bind the coated antigen. Anti-IDO2 Ig that bound the fixed protein was detected with an anti-mouse IgG FC-specific HRP secondary antibody and ABTS substrate.

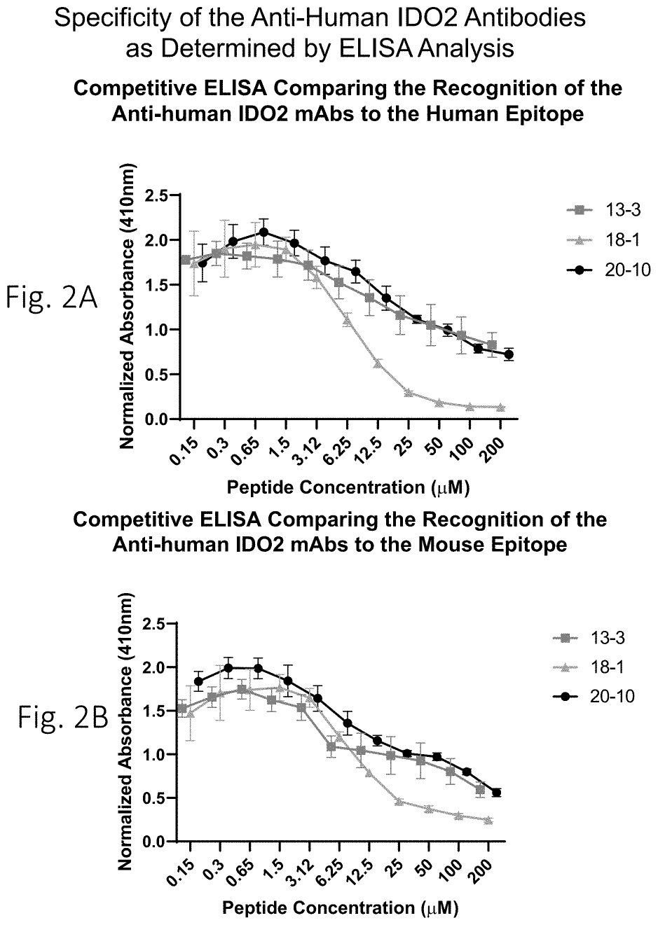

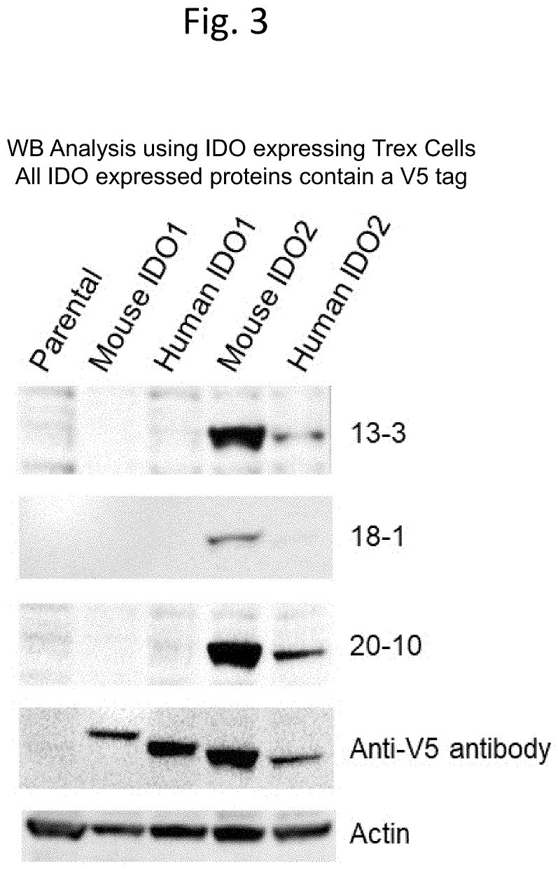

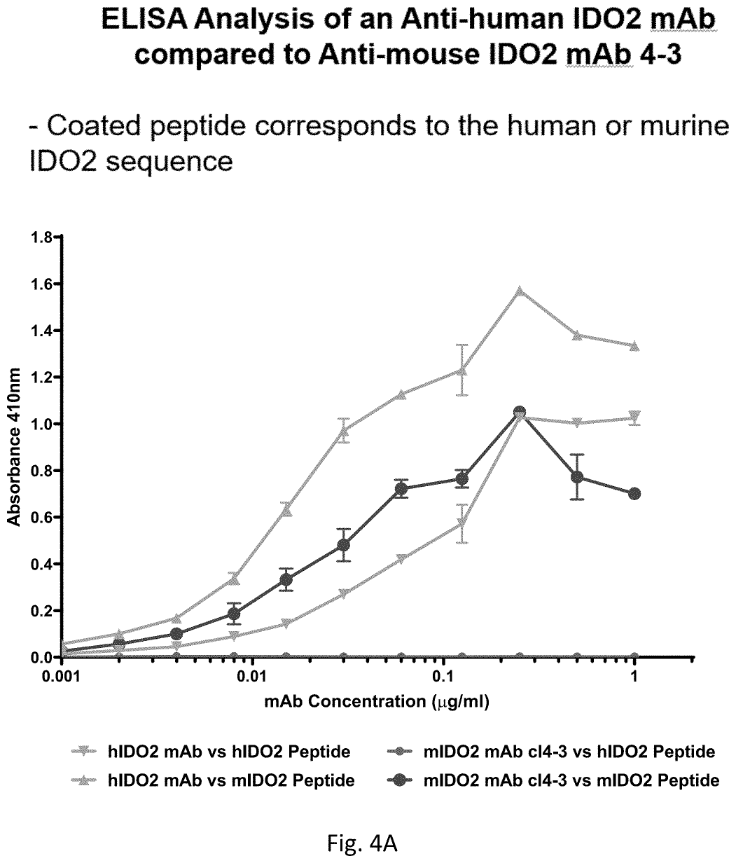

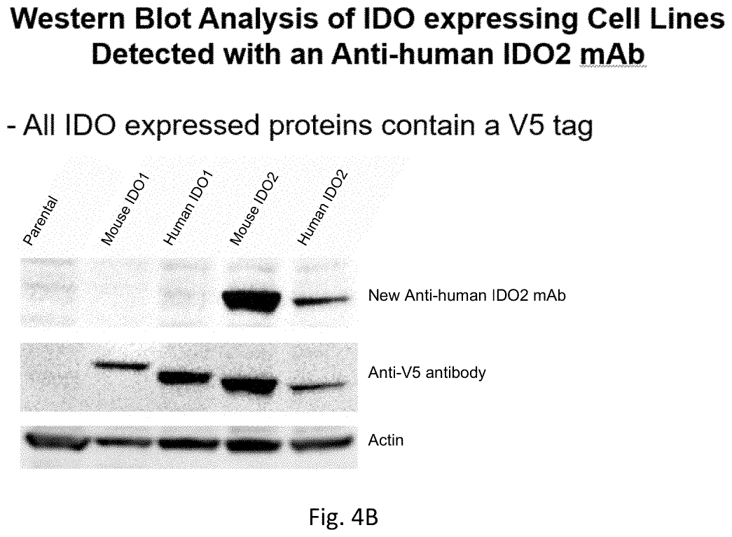

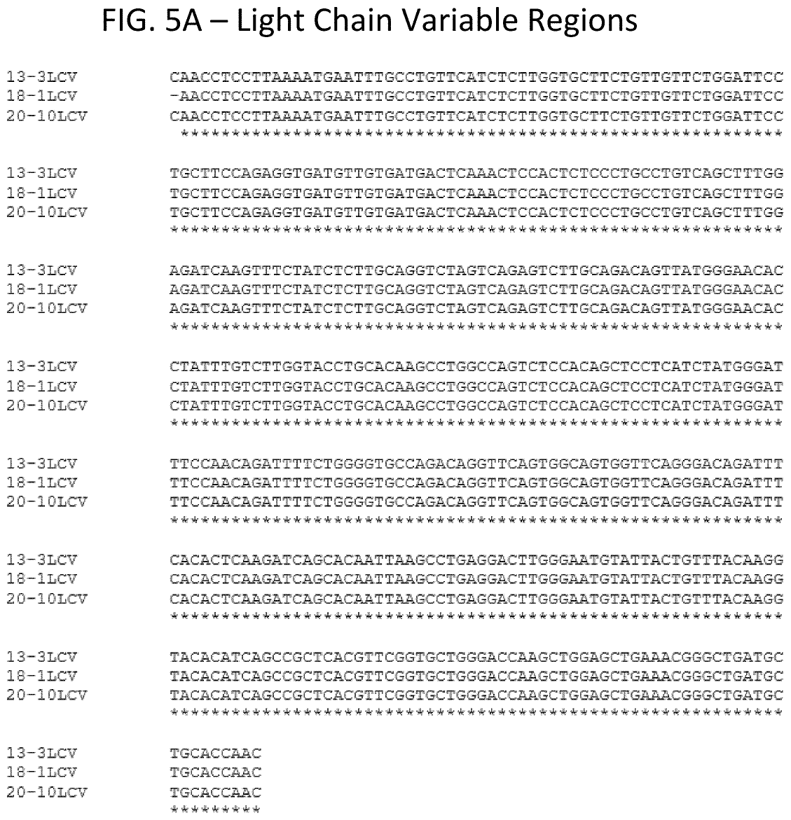

The results are shown in FIGS. 2A and 2B. These data show that the antibodies recognize human IDO2 protein, as well as the corresponding murine sequence. FIG. 2A is compares the recognition of the three anti-human IDO2 mAbs to the human epitope, plotting absorbance vs. epitope peptide concentration. FIG. 2B compares the recognition of the three anti-human IDO2 mAbs 1 to the mouse epitope, plotting absorbance vs. epitope peptide concentration. The anti-mouse IDO2 mAb was 4-3. The coated peptide corresponds to the human or murine IDO2 sequence.

### Example 3—Western Blot

A Western blot analysis was conducted using IDO-expressing Tᵣₑₓ cells. All IDO expressed proteins mouse and human IDO 1 and IDO2 containing a V5 tag. Western blot analysis demonstrates the specificity of the anti-human IDO2 antibody for human and mouse IDO2 and demonstrates that the same antibodies do not recognize human or murine IDO1. Cell extracts for IDO expressing cells or the parental T-REX line were prepared and fractionated as previously described (See US2019/0062452, incorporated by reference herein). Detection of indicated proteins was done using standard blotting methods, HRP conjugated secondary antibodies and chemiluminescence detection reagents.

Each and every patent, patent application, and publication, including publications listed herein and publicly available nucleic acid and amino acid sequences cited throughout the disclosure, is expressly incorporated herein by reference in its entirety. While this invention has been disclosed with reference to specific embodiments, it is apparent that other embodiments and variations of this invention are devised by others skilled in the art without departing from the true spirit and scope of the invention. The appended claims include such embodiments and equivalent variations.

### Example 4—Clinical Treatment

The information herein above can be applied clinically to patients for therapeutic intervention. A preferred embodiment of the invention comprises clinical application of the pharmaceutical compositions described herein to a patient. This can occur after a patient arrives in the clinic and presents with symptoms of an IDO2-related disease, as identified above.

The derived therapeutic dose of the anti-IDO2 antibody for an adult human may be determined by a physician, considering the patient's age, sex, weight, general medical condition, and the specific condition and severity thereof for which the antibody is being administered. The physician may also consider the route of administration of the antibody, the pharmaceutical carrier with which the antibody may be combined, and the antibody's biological activity.

The derived therapeutic does of the mRNA vaccines encoding the sequences described herein may be determined by a physician considering the patient's age, sex, weight, general medical condition, and the specific condition and severity thereof for which the antibody is being administered. The physician may also consider the route of administration of the vaccine, the pharmaceutical carrier with which the vaccine may be combined, and the vaccine's biological activity.

Anti-IDO2 antibodies and mRNA vaccines have been shown to be well tolerated and the symptoms were assessed using clinical scores criteria.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A recombinant, synthetic or monoclonal human antibody or antibody construct that binds to an epitope, or a fragment of said antibody that specifically or preferentially binds human IDO2, said antibody or construct comprising at least one heavy chain variable region CDR selected from SEQ ID NO: 16, 18 or 20.

2. The recombinant, synthetic or monoclonal human antibody or antibody construct according to claim 1, further comprising at least one heavy chain variable domain sequence that is at least 80% identical to an amino acid sequence selected from the group consisting of SEQ ID NOs. 10, 12 or 14.

3. The recombinant, synthetic or monoclonal human antibody or antibody construct according to claim 1, further comprising a light chain variable domain sequence that is at least 80% identical to an amino acid sequence selected from the group consisting of SEQ ID NO: 2.

4. The recombinant, synthetic or monoclonal human antibody or antibody construct according to claim 1, wherein said antibody is a chimeric antibody, a humanized antibody, a human antibody, a CDR-grafted antibody, a multi-specific binding construct that can bind two or more targets, a dual specific antibody, a bi-specific antibody or a multi-specific antibody, or an affinity matured antibody.

5. The recombinant, synthetic or monoclonal human antibody or antibody construct according to claim 1, wherein said fragment is a single-domain antibody (sdAb), a single antibody chain or an scFv fragment, a diabody, a single chain comprising complementary scFvs (tandem scFvs) or bispecific tandem scFvs, an Fv construct, a disulfide-linked Fv, a Fab construct, a Fab′ construct, a F(ab′)₂ construct, a monovalent or bivalent construct from which domains non-essential to monoclonal antibody function have been removed, a single-chain molecule containing one VL, one VH antigen-binding domain, and one or two constant “effector” domains optionally connected by linker domains, a univalent antibody lacking a hinge region, a single domain antibody, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, or a monobody.

6-8. (canceled)

9. The recombinant, synthetic or monoclonal human antibody or antibody construct according to claim 2, which is an IgG.

10. The recombinant, synthetic or monoclonal human antibody or antibody construct according to claim 9, which is an IgG1 isotype.

11. A recombinant, synthetic or monoclonal human antibody of an IgG class that binds to an epitope, or a fragment of said antibody that binds an epitope, said antibody comprising:
(a) a nucleotide sequence encoding a heavy chain variable domain sequence that is at least 80% identical to a nucleotide sequence selected from the group consisting of SEQ ID NOs. 9, 11 or 13; and
(b) a nucleotide sequence encoding a light chain variable domain sequence that is at least 80% identical to the nucleotide sequence of SEQ ID NOs: 1.

12. The antibody or epitope binding fragment according to claim 11, wherein the antibody comprises a heavy chain and light chain variable domain sequence encoded by SEQ ID NO: 9 and SEQ ID NO: 1 or sequences at least 80% identical thereto.

13. The antibody or epitope binding fragment according to claim 11, wherein the antibody comprises a heavy chain and light chain variable domain sequence encoded by SEQ ID NO: 11 and SEQ ID NO: 1 or sequences at least 80% identical thereto.

14. The antibody or epitope binding fragment according to claim 11, wherein the antibody comprises a heavy chain and light chain variable domain sequence encoded by SEQ ID NO: 13 and SEQ ID NO: 1 or sequences at least 80% identical thereto.

15. An antigen binding fragment thereof comprising one or more heavy chain CDR selected from the amino acid sequences SEQ ID NOS: 16, 18 or 20 and optionally one of more light chain CDR selected from SEQ ID NO: 4 or 7 or the sequence GIS.

16-20. (canceled)

21. The antibody or epitope binding fragment according to claim 1, wherein said antibody has the FR1/CDR1/FR2/CDR2/FR3CDR3/FR4 sequence combination of the heavy and light chain of mAb 20-10.

22. An isolated nucleic acid encoding the antibody or epitope according to claim 1.

23. The isolated nucleic acid according to claim 22, wherein said nucleic acid is an mRNA.

24. An mRNA vaccine comprising the mRNA according to claim 23.

25. (canceled)

26. A pharmaceutical composition for the prevention or treatment of an autoimmune disease comprising at least one antibody or epitope-binding fragment of claim 1.
